(19) United States
(12) Patent Application Publication
(10) Pub. No.: US 2023/0312856 A1
(43) Pub. Date: Oct. 5, 2023

(54) NANOPORE DEVICES INCLUDING BARRIERS USING DIBLOCK OR TRIBLOCK COPOLYMERS, AND METHODS OF MAKING THE SAME

(71) Applicant: Illumina Cambridge Limited, Cambridge (GB)

(72) Inventors: Alexandre Richez, Cambridge (GB); Antonio Conde-Gonzalez, Cambridge (GB); Oliver Uttley, Cambridge (GB); Charlotte Vacogne, Cambridge (GB); Davide Garoldini, Cambridge (GB); Istvan Kocsis, Cambridge (GB); Miguel Angel Aleman Garcia, Sawston (GB); Yuliia Vyborna, Cambridge (GB)

(73) Assignee: Illumina Cambridge Limited, Cambridge (GB)

(21) Appl. No.: 18/193,492

(22) Filed: Mar. 30, 2023

Related U.S. Application Data
(60) Provisional application No. 63/325,726, filed on Mar. 31, 2022.

(51) Int. Cl. C08J 9/228

(52) U.S. Cl. CPC C08J 9/228; C08J 2353/00; C08J 2205/042

(57) ABSTRACT

Nanopore devices including barriers using diblock or triblock copolymers, and methods of making the same, are provided herein. In some examples, a barrier between first and second fluids is suspended by a barrier support defining an aperture. The barrier may include one or more layers suspended across the aperture and including molecules of a block copolymer. Each molecule of the block copolymer may include one or more hydrophilic blocks having an approximate length A and one or more hydrophobic blocks having an approximate length B. The hydrophilic blocks may form outer surfaces of the barrier and the hydrophobic blocks may be located within the barrier. The hydrophobic blocks may include a polymer selected from the group consisting of poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

### CROSS-REFERENCE TO RELATED APPLICATIONS

This application claims the benefit of U.S. Provisional Patent Application No. 63/325,726, filed Mar. 31, 2022 and entitled “NANOPORE DEVICES INCLUDING BARRIERS USING DIBLOCK OR TRIBLOCK COPOLYMERS, AND METHODS OF MAKING THE SAME”, the entire contents of which are incorporated by reference herein.

### FIELD

This application relates to barriers that include block copolymers.

### BACKGROUND

A significant amount of academic and corporate time and energy has been invested into using nanopores to sequence polynucleotides. For example, the dwell time has been measured for complexes of DNA with the Klenow fragment (KF) of DNA polymerase I atop a nanopore in an applied electric field. Or, for example, a current or flux-measuring sensor has been used in experiments involving DNA captured in an α-hemolysin nanopore. Or, for example, KF-DNA complexes have been distinguished on the basis of their properties when captured in an electric field atop an α-hemolysin nanopore. In still another example, polynucleotide sequencing is performed using a single polymerase enzyme complex including a polymerase enzyme and a template nucleic acid attached proximal to a nanopore, and nucleotide analogs in solution. The nucleotide analogs include charge blockade labels that are attached to the polyphosphate portion of the nucleotide analog such that the charge blockade labels are cleaved when the nucleotide analog is incorporated into a polynucleotide that is being synthesized. The charge blockade label is detected by the nanopore to determine the presence and identity of the incorporated nucleotide and thereby determine the sequence of a template polynucleotide. In still other examples, constructs include a transmembrane protein pore subunit and a nucleic acid handling enzyme.

However, such previously known devices, systems, and methods may not necessarily be sufficiently robust, reproducible, or sensitive and may not have sufficiently high throughput for practical implementation, e.g., demanding commercial applications such as genome sequencing in clinical and other settings that demand cost effective and highly accurate operation. Accordingly, what is needed are improved devices, systems, and methods for sequencing polynucleotides, which may include using membranes having nanopores disposed therein.

### SUMMARY

Nanopore devices including barriers using diblock or triblock copolymers, and methods of making the same, are provided herein.

Some examples herein provide a barrier between first and second fluids. The barrier may be suspended by a barrier support defining an aperture. The barrier may include one or more layers suspended across the aperture and comprising molecules of a block copolymer. Each molecule of the block copolymer including one or more hydrophilic blocks having an approximate length A and one or more hydrophobic blocks having an approximate length B. The hydrophilic blocks may form outer surfaces of the barrier and the hydrophobic blocks may be located within the barrier. In some examples, the hydrophobic blocks include a polymer selected from the group consisting of poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

In some examples, the block copolymer is a diblock copolymer. In some examples, the hydrophobic block is polybutadiene (PBd). Additionally, or alternatively, in some examples, the barrier has a thickness of approximately 2A+2B.

In some examples, the block copolymer is a triblock copolymer having two hydrophilic blocks and one hydrophobic block. In some examples, the hydrophobic block is poly(isobutylene) (PIB). Additionally, or alternatively, in some examples, the barrier has a thickness of approximately 2A+B.

In some examples, the block copolymer is a triblock copolymer having two hydrophobic blocks and one hydrophilic block. In some examples, the barrier has a thickness of approximately A+2B.

In some examples, the barrier further includes including a nanopore disposed therein and providing contact between the first fluid and the second fluid.

Some examples herein provide a barrier between first and second fluids. The barrier may be suspended by a barrier support defining an aperture. The barrier may include one or more layers suspended across the aperture and including molecules of a block copolymer. Each molecule of the block copolymer may include first and second hydrophilic blocks and a hydrophobic block disposed between the first and second hydrophilic blocks. The first and second hydrophilic blocks may form outer surfaces of the barrier and the hydrophobic blocks may be located within the barrier. The barrier further may include a nanopore disposed within the barrier and providing contact between the first fluid and the second fluid.

In some examples, the first and second hydrophilic blocks are approximately of length A, the hydrophobic block is approximately of length B, at least a portion of the one or more layers has a thickness of approximately 2A+B, and the barrier has a thickness of approximately 2A+B. In some examples, the length A is about 2 repeating units to about 100 repeating units. Additionally, or alternatively, the length B is about 2 repeating units to about 100 repeating units.

In some examples, the hydrophobic block includes a polymer selected from the group consisting of poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

In some examples, the first and second hydrophilic blocks include a polymer selected from the group consisting of: N-vinyl pyrrolidone, polyacrylamide, zwitterionic polymer, polypeptide, and poly(ethylene oxide) (PEO).

In some examples, the hydrophobic block includes a polymer having a glass transition temperature (T_{g}) of less than about 0° C.

In some examples, at least one of the first and second hydrophilic blocks includes a moiety selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen.

In some examples, the moiety of the first hydrophilic block includes a methyl group or a carboxyl group, and wherein the moiety of the second hydrophilic block includes a methyl group or a carboxyl group.

In some examples, the molecules further include a first linker coupling the first hydrophilic block to a first end of the hydrophobic block, and a second linker coupling the second hydrophilic block to a second end of the hydrophobic block.

Some examples herein provide a barrier between first and second fluids. The barrier may include a first layer including a first plurality of molecules of a diblock copolymer. Each molecule of the diblock copolymer may include a hydrophobic block coupled to a hydrophilic block. The barrier further may include a second layer including a second plurality of molecules of the diblock copolymer. The hydrophilic blocks of the first plurality of molecules may form a first outer surface of the barrier, the hydrophilic blocks of the second plurality of molecules may form a second outer surface of the barrier, and the hydrophobic blocks of the first and second pluralities of molecules may contact one another within the barrier.

In some examples, the hydrophilic block is approximately of length A, the hydrophobic block is approximately of length B, the first and second layers each have a thickness of approximately A+B, and the barrier has a thickness of approximately 2A+2B. In some examples, the length A is about 2 repeating units to about 100 repeating units. Additionally, or alternatively, the length B is about 2 repeating units to about 100 repeating units.

In some examples, the hydrophilic block includes a polymer selected from the group consisting of: N-vinyl pyrrolidone, polyacrylamide, zwitterionic polymer, polypeptide, and poly(ethylene oxide) (PEO).

In some examples, the hydrophobic block includes a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

In some examples, each of the molecules further includes a linker coupling the hydrophobic block to the hydrophilic block.

In some examples, the linker includes at least one moiety selected from the group consisting of: an amide, a thioether (sulfide), a succinic group, a maleic group, a methylene, an ether, and a product of a click reaction.

In some examples, the first and second hydrophilic blocks each include a moiety independently selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen.

In some examples, the barrier further includes a nanopore disposed within the barrier and providing an aperture fluidically coupling the first fluid to the second fluid.

Some examples herein provide a barrier between first and second fluids. The barrier may include a first layer including a first plurality of molecules of a triblock copolymer. Each molecule of the triblock copolymer may include first and second hydrophobic blocks and a hydrophilic block disposed between the first and second hydrophobic blocks. The barrier further may include a second layer including a second plurality of molecules of the triblock copolymer. The hydrophilic blocks of the first plurality of molecules may form a first outer surface of the barrier, the hydrophilic blocks of the second plurality of molecules may form a second outer surface of the barrier, and the hydrophobic blocks of the first and second pluralities of molecules may contact one another within the barrier.

In some examples, the hydrophilic block is approximately of length A, each hydrophobic block is approximately of length B, the first and second layers each have a thickness of approximately A/2+B, and the barrier has a thickness of approximately A+2B. In some examples, the length A is about 2 repeating units to about 100 repeating units. Additionally, or alternatively, the length B is about 2 repeating units to about 100 repeating units.

In some examples, the hydrophilic block includes a polymer selected from the group consisting of: N-vinyl pyrrolidone, polyacrylamide, zwitterionic polymer, polypeptide, and poly(ethylene oxide) (PEO).

In some examples, the hydrophobic blocks each include a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

In some examples, each of the molecules of the first and second plurality of molecules further includes a linker coupling each hydrophobic block to the hydrophilic block. In some examples, the linker is selected from the group consisting of an amide, a thioether (sulfide), a succinic group, a maleic group, a methylene, an ether, and a product of a click reaction.

In some examples, the hydrophilic block includes a moiety selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen.

In some examples, the barrier further includes a nanopore disposed within the barrier and providing an aperture fluidically coupling the first fluid to the second fluid.

Some examples herein provide a method of forming a barrier. The method may include forming a barrier between first and second fluids, the barrier being suspended by a barrier support defining an aperture. The barrier may include one or more layers suspended across the aperture and including molecules of a block copolymer. Each molecule of the block copolymer may include one or more hydrophilic blocks having an approximate length A and one or more hydrophobic blocks having an approximate length B. The hydrophilic blocks may form outer surfaces of the barrier and the hydrophobic blocks may be located within the barrier. In some examples, the one or more hydrophobic blocks include a polymer selected from the group consisting of poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

In some examples, the barrier is formed using painting. In some examples, the painting is selected from the group consisting of brush painting, mechanical painting, and bubble painting.

In some examples, the one or more hydrophilic blocks include a polymer selected from the group consisting of: N-vinyl pyrrolidone, polyacrylamide, zwitterionic polymer, polypeptide, and poly(ethylene oxide) (PEO).

In some examples, the method further includes inserting a nanopore into the barrier. In some examples, the nanopore provides contact between the first fluid and the second fluid. Additionally, or alternatively, in some examples, the nanopore may be inserted into the barrier electroporation, pipette pump cycle, or detergent assisted pore insertion.

In some examples, the block copolymer is a diblock copolymer.

In some examples, the block copolymer is a triblock copolymer having two hydrophilic blocks and one hydrophobic block.

In some examples, the block copolymer is a triblock copolymer having two hydrophobic blocks and one hydrophilic block.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1 schematically illustrates a cross-sectional view of an example nanopore composition and device including a barrier using a diblock or triblock copolymer.

FIGS. 2A-2C schematically illustrate barriers using diblock or triblock copolymers which may be included in the nanopore composition and device of FIG. 1.

FIGS. 3A-3C schematically illustrate example schemes for preparing triblock copolymers for use in the nanopore composition and device of FIG. 1.

FIG. 4 schematically illustrates a cross-sectional view of an example use of the composition and device of FIG. 1.

FIG. 5 schematically illustrates a cross-sectional view of another example use of the composition and device of FIG. 1.

FIG. 6 schematically illustrates a cross-sectional view of another example use of the composition and device of FIG. 1.

FIG. 7 schematically illustrates a cross-sectional view of another example use of the composition and device of FIG. 1.

FIG. 8 illustrates a flow of operations for forming a device such as illustrated in FIG. 1.

FIG. 9 illustrates plots and a table describing capacitance and noise measured for membranes formed from DPhPC, PEO₅₀₀-b-PBd₇₀₀, and PDMS₅₀₀-b-PEO₄₂₀-b-PDMS500.

FIG. 10 illustrates a plot of the normalized number of remaining membranes as a function of measured voltage for DPhPC, PEO₅₀₀-b-PBd₇₀₀, and PDMS₅₀₀-b-PEO₄₂₀-b-PDMS₅₀₀ membranes.

FIG. 11 illustrates a plot of MspA nanopore/PEO₅₀₀-b-PBd₇₀₀ membrane construct stability in 1M KCl+50 mM HEPES buffer.

FIG. 12 illustrates a plot describing the breakdown voltage measured for membranes formed in accordance with examples herein.

FIG. 13 illustrates a plot of MspA nanopore/membrane construct stability under certain conditions.

FIGS. 14A-14B schematically illustrate plan and cross-sectional views of further details of one nonlimiting example of the nanopore composition and device of FIG. 1.

FIG. 15 schematically illustrates an alternative barrier that may be used in the example described with reference to FIGS. 14A-14B.

FIG. 16 schematically illustrates another alternative barrier that may be used in the example described with reference to FIGS. 14A-14B.

FIG. 17 schematically illustrates a cross-sectional view of another example use of the composition and device of FIG. 1.

### DETAILED DESCRIPTION

Nanopore devices including barriers using diblock or triblock copolymers, and methods of making the same, are provided herein.

For example, nanopore sequencing may utilize a nanopore that is inserted into a barrier, and that includes an aperture through which ions and/or other molecules may flow from one side of the barrier to the other. Circuitry may be used to detect a sequence of nucleotides, e.g., during sequencing-by-synthesis (SBS) in which, on a first side of the barrier, a polymerase adds the nucleotides to a growing polynucleotide in an order that is based on the sequence of a template polynucleotide to which the growing polynucleotide is hybridized. The sensitivity of the circuitry may be improved by using fluids with different compositions on respective sides of the barrier, for example to provide suitable electron transport for detection on one side of the barrier, while suitably promoting activity of the polymerase on the other side of the barrier. The difference in fluidic compositions may generate an osmotic pressure that may weaken the barrier, and thus increase the likelihood that the barrier may break or leak during normal use. However, it may be difficult to insert nanopores into barriers that are too strong.

As provided herein, barriers for use in nanopore devices may include diblock or triblock copolymers that provide suitable stability characteristics for long-term use of the device, and that also facilitate nanopore insertion so as to increase the number of usable devices during production. As explained in greater detail below, in some examples, the present diblock copolymers may include a hydrophilic block coupled to a hydrophobic block. The respective lengths of the hydrophobic and hydrophilic blocks in the diblock copolymer may be selected such that the copolymers assemble into a bilayer structure in which the hydrophilic block forms the outer surface of the respective layer (and thus a respective outer surface of the barrier), and the hydrophobic block forms the inner surface of that layer and contacts the hydrophobic block of the other layer. In other examples, the present triblock copolymers may include a hydrophilic block coupled between two hydrophobic blocks. The respective lengths of the hydrophobic and hydrophilic blocks in the triblock copolymer may be selected such that the copolymers assemble into a bilayer structure in which the hydrophilic block forms the outer surface of the respective layer (and thus a respective outer surface of the barrier), and the hydrophobic block forms the inner surface of that layer and contacts the hydrophobic block of the other layer. In still other examples, the present triblock copolymers may include a hydrophobic block coupled between two hydrophilic blocks. The respective lengths of the hydrophobic and hydrophilic blocks in the triblock copolymer may be selected such that the copolymers assemble into a structure in which the hydrophilic blocks form the outer surface of the barrier, and the hydrophobic block forms the inside of the barrier.

First, some terms used herein will be briefly explained. Then, some example devices including barriers using diblock or triblock copolymers, and methods of making and using the same, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or system, the term “comprising” means that the compound, composition, or system includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, Calif.).

As used herein, the term “electrode” is intended to mean a solid structure that conducts electricity. Electrodes may include any suitable electrically conductive material, such as gold, palladium, silver, or platinum, or combinations thereof. In some examples, an electrode may be disposed on a substrate. In some examples, an electrode may define a substrate.

As used herein, the term “nanopore” is intended to mean a structure that includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. Molecules that can cross through an aperture of a nanopore can include, for example, ions or water-soluble molecules such as amino acids or nucleotides. The nanopore can be disposed within a barrier, or can be provided through a substrate. Optionally, a portion of the aperture can be narrower than one or both of the first and second sides of the nanopore, in which case that portion of the aperture can be referred to as a “constriction.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions. nanopores include biological nanopores, solid-state nanopores, or biological and solid-state hybrid nanopores.

Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer or a heteropolymer. Structures of polypeptide nanopores include, for example, an α-helix bundle nanopore and a β-barrel nanopore as well as all others well known in the art. Example polypeptide nanopores include aerolysin, α-hemolysin, Mycobacterium smegmatis porin A, gramicidin A, maltoporin, OmpF, OmpC, PhoE, Tsx, F-pilus, SP1, mitochondrial porin (VDAC), Tom40, outer membrane phospholipase A, CsgG, and Neisseria autotransporter lipoprotein (NaIP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105: 20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Norcadia farcinica, and lysenin. For further details regarding lysenin, see PCT Publication No. WO 2013/153359, the entire contents of which are incorporated by reference herein.

A “polynucleotide nanopore” is intended to mean a nanopore that is made from one or more nucleic acid polymers. A polynucleotide nanopore can include, for example, a polynucleotide origami.

A “solid-state nanopore” is intended to mean a nanopore that is made from one or more materials that are not of biological origin. A solid-state nanopore can be made of inorganic or organic materials. Solid-state nanopores include, for example, silicon nitride (SiN), silicon dioxide (SiO₂), silicon carbide (SiC), hafnium oxide (HfO₂), molybdenum disulfide (MoS₂), hexagonal boron nitride (h-BN), or graphene. A solid-state nanopore may comprise an aperture formed within a solid-state membrane, e.g., a membrane including any such material(s).

A “biological and solid-state hybrid nanopore” is intended to mean a hybrid nanopore that is made from materials of both biological and non-biological origins. Materials of biological origin are defined above and include, for example, polypeptides and polynucleotides. A biological and solid-state hybrid nanopore includes, for example, a polypeptide-solid-state hybrid nanopore and a polynucleotide-solid-state nanopore.

As used herein, a “barrier” is intended to mean a structure that normally inhibits passage of molecules from one side of the barrier to the other side of the barrier. The molecules for which passage is inhibited can include, for example, ions or water soluble molecules such as nucleotides and amino acids. However, if a nanopore is disposed within a barrier, then the aperture of the nanopore may permit passage of molecules from one side of the barrier to the other side of the barrier. As one specific example, if a nanopore is disposed within a barrier, the aperture of the nanopore may permit passage of molecules from one side of the barrier to the other side of the barrier. Barriers include membranes of biological origin, such as lipid bilayers, and non-biological barriers such as solid-state membranes or substrates.

As used herein, “of biological origin” refers to material derived from or isolated from a biological environment such as an organism or cell, or a synthetically manufactured version of a biologically available structure.

As used herein, “solid-state” refers to material that is not of biological origin.

As used herein, “synthetic” refers to a membrane material that is not of biological origin (e.g., polymeric materials, synthetic phospholipids, solid-state membranes, or combinations thereof).

As used herein, a “solution” is intended to refer to a homogeneous mixture including two or more substances. In such a mixture, a solute is a substance which is dissolved in another substance referred to as a solvent. A solution may include a single solute, or may include a plurality of solutes. An “aqueous solution” refers to a solution in which the solvent is, or includes, water.

As used herein, a “polymeric membrane” or a “polymer membrane” refers to a synthetic barrier that primarily is composed of a polymer that is not of biological origin. In some examples, a polymeric membrane consists essentially of a polymer that is not of biological origin. A block copolymer is an example of a polymer that is not of biological origin and that may be included in the present barriers. A hydrophobic polymer with ionic end groups is another example of a polymer that is not of biological origin and that may be included in the present barriers. Because the present barriers relate to polymers that are not of biological origin, the terms “polymeric membrane,” “polymer membrane,” “membrane,” and “barrier” may be used interchangeably herein when referring to the present barriers, even though the terms “barrier” and “membrane” generally may encompass other types of materials as well.

As used herein, the term “block copolymer” is intended to refer to a polymer having at least a first portion or “block” that includes a first type of monomer, and at least a second portion or “block” that is coupled directly or indirectly to the first portion and includes a second, different type of monomer. The first portion may include a polymer of the first type of monomer, or the second portion may include a polymer of the second type of monomer, or the first portion may include a polymer of the first type of monomer and the second portion may include a polymer of the second type of monomer. The first portion optionally may include an end group with a hydrophilicity that is different than that of the first type of monomer, or the second portion optionally may include an end group with a hydrophilicity that is different than that of the second type of monomer, or the first portion optionally may include an end group with a hydrophilicity that is different than that of the first type of monomer and the second portion optionally may include an end group with a hydrophilicity that is different than that of the second type of monomer. The end groups of any hydrophilic blocks may be located at an outer surface of a barrier formed using such hydrophilic blocks. Depending on the particular configuration, the end groups of any hydrophobic blocks may be located at an inner surface of the barrier or at an outer surface of a barrier formed using such hydrophobic blocks.

Block copolymers include, but are not limited to, diblock copolymers and triblock copolymers.

A “diblock copolymer” is intended to refer to a block copolymer that includes, or consists essentially of, first and second blocks coupled directly or indirectly to one another. The first block may be hydrophilic and the second block may be hydrophobic, in which case the diblock copolymer may be referred to as an “AB” copolymer where “A” refers to the hydrophilic block and “B” refers to the hydrophobic block.

A “triblock copolymer” is intended to refer to a block copolymer that includes, or consists essentially of, first, second, and third blocks coupled directly or indirectly to one another. The first and third blocks may include, or may consist essentially of, the same type of monomer (repeating unit) as one another, and the second block may include a different type of monomer (repeating unit). In some examples, the first block may be hydrophobic, the second block may be hydrophilic, and the third block may be hydrophobic and includes the same type of monomer as the first block, in which case the triblock copolymer may be referred to as a “BAB” copolymer where “A” refers to the hydrophilic block and “B” refers to the hydrophobic blocks. In other examples, the first block may be hydrophilic, the second block may be hydrophobic, and the third block may be hydrophilic and includes the same type of monomer as the first block, in which case the triblock copolymer may be referred to as an “ABA” copolymer where “A” refers to the hydrophilic blocks and “B” refers to the hydrophobic block.

The particular arrangement of molecules of polymer chains (e.g., block copolymers) within a polymeric membrane may depend, among other things, on the respective block lengths, the type(s) of monomers used in the different blocks, the relative hydrophilicities and hydrophobicities of the blocks, the composition of the fluid(s) within which the membrane is formed, and/or the density of the polymeric chains within the membrane. During formation of the membrane, these and other factors generate forces between molecules of the polymeric chains which laterally position and reorient the molecules in such a manner as to substantially minimize the free energy of the membrane. The membrane may be considered to be substantially “stable” once the polymeric chains have completed these rearrangements, even though the molecules may retain some fluidity of movement within the membrane.

As used herein, the term “hydrophobic” is intended to mean tending to exclude water molecules. Hydrophobicity is a relative concept relating to the polarity difference of molecules relative to their environment. Non-polar (hydrophobic) molecules in a polar environment will tend to associate with one another in such a manner as to reduce contact with polar (hydrophilic) molecules to a minimum to lower the free energy of the system as a whole.

As used herein, the term “hydrophilic” is intended to mean tending to bond to water molecules. Polar (hydrophilic) molecules in a polar environment will tend to associate with one another in such a manner as to reduce contact with non-polar (hydrophobic) molecules to a minimum to lower the free energy of the system as a whole.

As used herein, the term “amphiphilic” is intended to mean having both hydrophilic and hydrophobic properties. For example, a block copolymer that includes a hydrophobic block and a hydrophilic block may be considered to be “amphiphilic.” Illustratively, AB copolymers, ABA copolymers, and BAB copolymers all may be considered to be amphiphilic.

As used herein, the term “linker” is intended to mean a moiety, molecule, or molecules via which one element is attached to another element. Linkers may be covalent, or may be non-covalent. Nonlimiting examples of covalent linkers include moieties such as alkyl chains, polyethers, amides, esters, aryl groups, polyaryls, and the like. Nonlimiting examples of noncovalent linkers include host-guest complexation, cyclodextrin/norbornene, adamantane inclusion complexation with β-CD, DNA hybridization interactions, streptavidin/biotin, and the like.

As used herein, the terms “PEO”, “PEG”, “poly(ethylene oxide)”, and “poly(ethylene glycol)” are intended to be used interchangeably and refer to a polymer that comprises —[CH₂—CH₂—O]ₙ—. In some examples, n is between about 2 and about 100.

As used herein, the term “barrier support” is intended to refer to a structure that can suspend a barrier. A barrier support may define an aperture, such that a first portion of the barrier is suspended across the aperture, and a second portion of the barrier is disposed on, and supported by, the barrier. The barrier support may include any suitable arrangement of elements to define an aperture and suspend the barrier across the aperture. In some examples, a barrier support may include a substrate having an aperture defined therethrough, across which aperture the barrier may be suspended. Additionally, or alternatively, the barrier support may include one or more first features (such as one or more lips or ledges of a well within a substrate) that are raised relative to one or more second features (such as a bottom surface of the well), wherein a height difference between (a) the one or more first features and (b) the one or more second features defines an aperture across which a barrier may be suspended. The aperture may have any suitable shape, such as a circle, an oval, a polygon, or an irregular shape. The barrier support may include any suitable material or combination of materials. For example, the barrier support may be of biological origin, or may be solid state. Some examples, the barrier support may include, or may consist essentially of, an organic material, e.g., a curable resin such as SU-8; polytetrafluoroethylene (PTFE), poly(methyl methacrylate) (PMMA), parylene, or the like. Additionally, or alternatively, various examples, the barrier support may include, or may consist essentially of, an inorganic material, e.g., silicon nitride, silicon oxide, or molybdenum disulfide.

As used herein, the term “annulus” is intended to refer to a liquid that is adhered to a barrier support, located within a barrier, and extends partially into an aperture defined by the barrier support. As such, it will be understood that the annulus may follow the shape of the aperture of the barrier, e.g., may have the shape of a circle, an oval, a polygon, or an irregular shape.

### Nanopore Devices including Barriers Using Diblock or Triblock Copolymers, and Methods of Making the Same

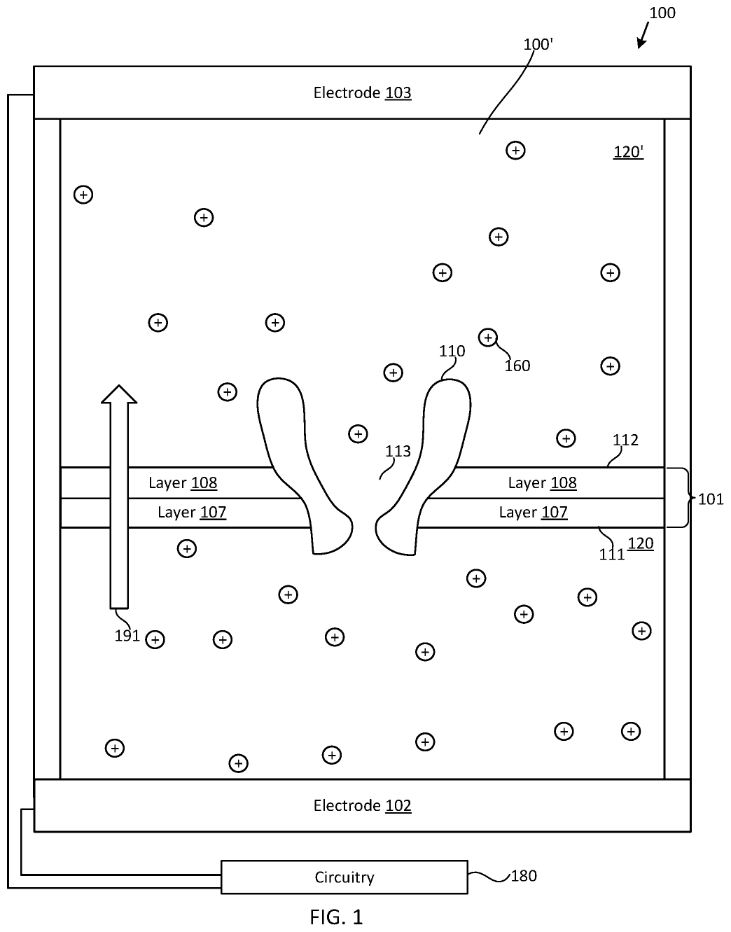

Some example devices including barriers using diblock or triblock copolymers, and methods of making the same, will be described with FIGS. 1, 2A-2C, 3A-3C, 4, 5, 6, 7, 8, 14A-14B, 15, 16, and 17.

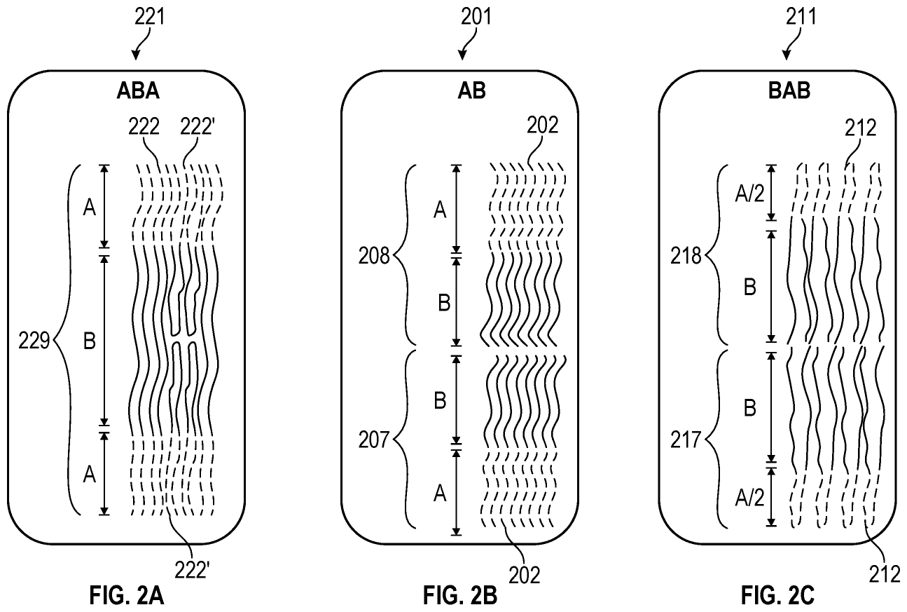

FIG. 1 schematically illustrates a cross-sectional view of an example nanopore composition and device 100 including a barrier using a diblock or triblock copolymer. Device 100 includes fluidic well 100′ including barrier 101 having first (trans) side 111 and second (cis) side 112, first fluid 120 within fluidic well 100′ and in contact with first side 111 of the barrier, and second fluid 120′ within the fluidic well and in contact with the second side 112 of the barrier. Barrier 101 may have any suitable structure that normally inhibits passage of molecules from one side of the barrier to the other side of the barrier, e.g., that normally inhibits contact between fluid 120 and fluid 120′. For example, as illustrated in FIG. 1, barrier 101 optionally may include first layer 107 and second layer 108, one or both of which inhibit the flow of molecules across that layer. Illustratively, barrier 101 may include a bilayer including layers 107 and 108 which respectively may be formed using AB diblock copolymers provided herein, or BAB triblock copolymers provided herein, or certain ABA triblock copolymers provided herein, and may have a structure such as described in greater detail below with reference to FIGS. 2A-2C, 14A-14B, 15, or 16. Alternatively, barrier 101 may include only a single layer, which inhibits the flow of molecules across that layer. Illustratively, barrier 101 may include a single layer which may be formed using certain ABA triblock copolymers provided herein, and may have a structure such as described in greater detail below with reference to FIG. 2A and FIG. 15. In other examples, barrier 101 may be partially a single layer, and partially a bilayer, formed using certain ABA triblock copolymers provided herein, and may have a structure such as described in greater detail below with reference to FIG. 2A and FIG. 15.

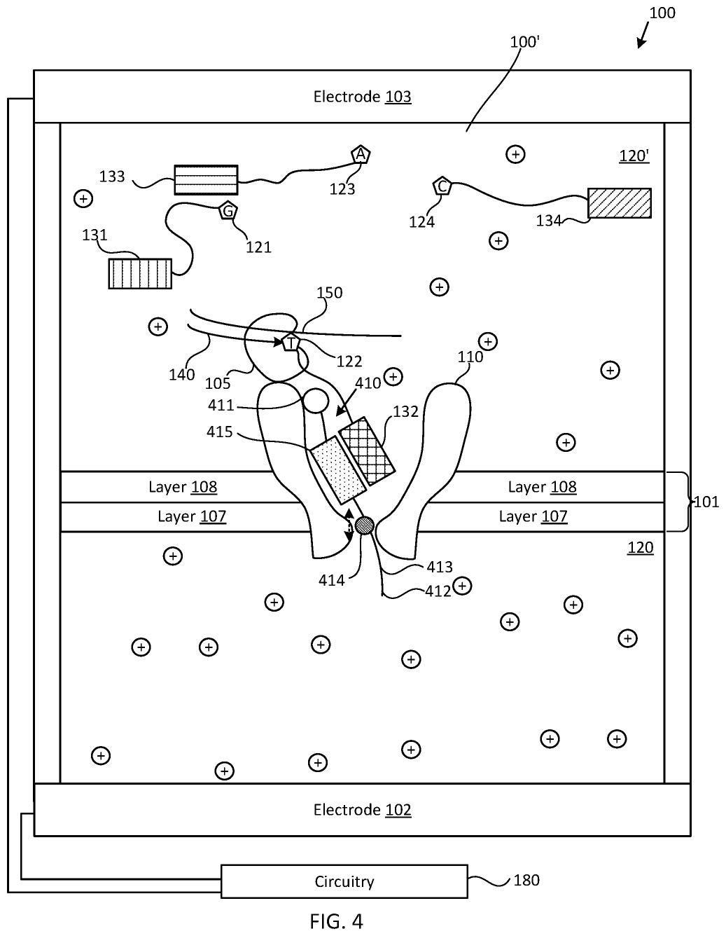

First fluid 120 may have a first composition including a first concentration of a salt 160, which salt may be represented for simplicity as positive ions although it will be appreciated that counterions also may be present. Second fluid 120′ may have a second composition including a second concentration of the salt 160 that may be the same as, or different, than the first concentration. Any suitable salt or salts 160 may be used in first and second fluids 120, 120′, e.g., ranging from common salts to ionic crystals, metal complexes, ionic liquids, or even water soluble organic ions. For example, the salt may include any suitable combination of cations (such as, but not limited to, H, Li, Na, K, NH₄, Ag, Ca, Ba, and/or Mg) with any suitable combination of anions (such as, but not limited to, OH, Cl, Br, I, NO₃, ClO₄, F, SO₄, and/or CO₃²⁻ . . . ). In one nonlimiting example, the salt includes potassium chloride (KCl). It will also be appreciated that the first and second fluids optionally may include any suitable combination of other solutes. Illustratively, first and second fluids 120, 120′ may include an aqueous buffer (such as N-(2-hydroxyethyl)piperazine-N′-2-ethanesulfonic acid (HEPES), commercially available from Fisher BioReagents). Any difference between the first and second concentrations of salt 160 and/or between other components of the first and second fluids, may generate osmotic pressure 191 across barrier 101. As provided herein, the diblock or triblock copolymer used in barrier 101 may be selected so as to provide the barrier with sufficient stability for use over a desired period of time, e.g., for use over the course of sequencing a polynucleotide in a manner such as described with reference to FIG. 4-7 or 17.

Still referring to FIG. 1, in some examples provided herein, device 100 optionally further may include nanopore disposed within barrier 101 and providing aperture 113 fluidically coupling first side 111 to second side 112. As such, aperture 113 of nanopore 110 may provide a pathway for fluid 120 and/or fluid 120′ to flow through barrier 101. For example, a portion of salt 160 may move from second side 112 of barrier 101 to first side 111 of the barrier through aperture 113. Nanopore 110 may include a solid-state nanopore, a biological nanopore (e.g., MspA such as illustrated in FIG. 1), or a biological and solid state hybrid nanopore. Nonlimiting examples and properties of barriers and nanopores are described elsewhere herein, as well as in U.S. Pat. No. 9,708,655, the entire contents of which are incorporated by reference herein. In a manner such as illustrated in FIG. 1, device 100 optionally may include first electrode 102 in contact with first fluid 120, second electrode 103 in contact with second fluid 120′, and circuitry 180 in operable communication with the first and second electrodes and configured to detect changes in an electrical characteristic of the aperture. Such changes may, for example, be responsive to any suitable stimulus. Indeed, it will be appreciated that the present methods, compositions, and devices may be used in any suitable application or context, including any suitable method or device for polynucleotide sequencing.

In some examples, first layer 107 of barrier 101 between first and second fluids 120, 120′ includes a first plurality of molecules of a diblock or triblock copolymer, and second layer 108 of barrier 101 includes a second plurality of molecules of the copolymer. In examples in which the copolymer is a diblock copolymer (which may be referred to as AB), each molecule may include a hydrophobic block coupled to a hydrophilic block. In some examples in which the copolymer is a triblock copolymer, each molecule may include first and second hydrophobic blocks and a hydrophilic block disposed therebetween (which polymer may be referred to as BAB). Regardless of whether the copolymer is diblock (AB) or triblock (BAB), the hydrophilic blocks of the first plurality of molecules may form a first outer surface of barrier 101, e.g., the surface of layer 107 contacting fluid 120 on first side 111. The hydrophilic blocks of the second plurality of molecules may form a second outer surface of barrier 101, e.g., the surface of layer 108 contacting fluid 120′ on second side 112. The hydrophobic blocks of the first and second pluralities of molecules may contact one another within the barrier.

In other examples, barrier 101 between first and second fluids 120, 120′ includes a plurality of molecules of a triblock copolymer that includes first and second hydrophilic blocks and a hydrophobic block disposed therebetween (which polymer may be referred to as ABA). The first hydrophilic blocks of the plurality of molecules may form a first outer surface of barrier 101, e.g., the surface of the barrier 101 contacting fluid 120 on first side 111. The second hydrophilic blocks of the plurality of molecules may form a second outer surface of barrier 101, e.g., contacting fluid 120′ on second side 112. The hydrophobic blocks of the plurality of molecules may contact one another within the barrier. In some examples, the ABA molecules that form barrier 101 may be present in a single layer rather than the two layers illustrated in FIG. 1. In other examples, the ABA molecules that form barrier 101 may be present in a bilayer similar to that illustrated in FIG. 1, because the molecules of the B blocks of the molecules are folded such that both A blocks of a given molecule contact the same fluid as one another. In still other examples, a portion of barrier 101 may be a single layer (because the ABA molecules of that portion extend from one fluid to the other fluid) while a different portion of barrier 101 may be a bilayer (because the ABA molecules of that portion are folded so the A blocks of that portion contact the same fluid as one another); FIG. 2A illustrates an example of this.

For example, FIGS. 2A-2C schematically illustrate barriers using diblock or triblock copolymers which may be included in the nanopore composition and device of FIG. 1. It will be appreciated that such barriers suitably may be adapted for use in any other composition or device, and are not limited to use with nanopores.

Referring now to FIG. 2A, barrier 221 uses a triblock “ABA” copolymer. Barrier 221 includes layer 229 which may contact both fluids 120 and 120′. Layer 229 includes a plurality of molecules 222 of a triblock ABA copolymer. As illustrated in FIG. 2A, each molecule 222 of the triblock copolymer includes first and second hydrophilic blocks, each denoted “A” and being approximately of length “A,” and a hydrophobic block disposed between the first and second hydrophilic blocks, denoted “B” and being approximately of length “B”. The hydrophilic A blocks at first ends of molecules 222 (the molecules forming layer 229) form a first outer surface of the barrier 221, e.g., contact fluid 120. The hydrophilic A blocks at second ends of molecules 222 form a second outer surface of the barrier 221, e.g., contact fluid 120′. The hydrophobic B blocks of the molecules 222 are within the barrier 211 in a manner such as illustrated in FIG. 2C. As illustrated, the majority of molecules 222 within layer 229 may extend substantially linearly and in the same orientation as one another. Optionally, as illustrated in FIG. 2A, some of the molecules 222′ may be folded at their B blocks, such that both of the hydrophilic A blocks of such molecules may contact the same fluid as one another. Accordingly, the example shown in FIG. 2A may be considered to be partially a single layer, and partially a bilayer. In other examples (not specifically illustrated), layer 229 may be entirely a single-layer or may be entirely a bilayer, e.g., as also described with reference to FIG. 1. Regardless of whether the membrane includes molecules 222 which extend substantially linearly and/or molecules 222′ which are folded, as illustrated in FIG. 2A, layer 229 may have a thickness of approximately 2A+B. In some examples, length A is about 1 RU to about 100 RU, e.g., about 2 RU to about 100 RU, or about 10 RU to about 80 RU, or about 20 RU to about 50 RU, or about 50 RU to about 80 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU. It will be appreciated that any end groups that are coupled to the hydrophilic or hydrophobic blocks contribute to the overall thickness of the barrier. Optionally, barrier 221 described with reference to FIG. 2A may be suspended across an aperture in a manner such as described with reference to FIG. 15.

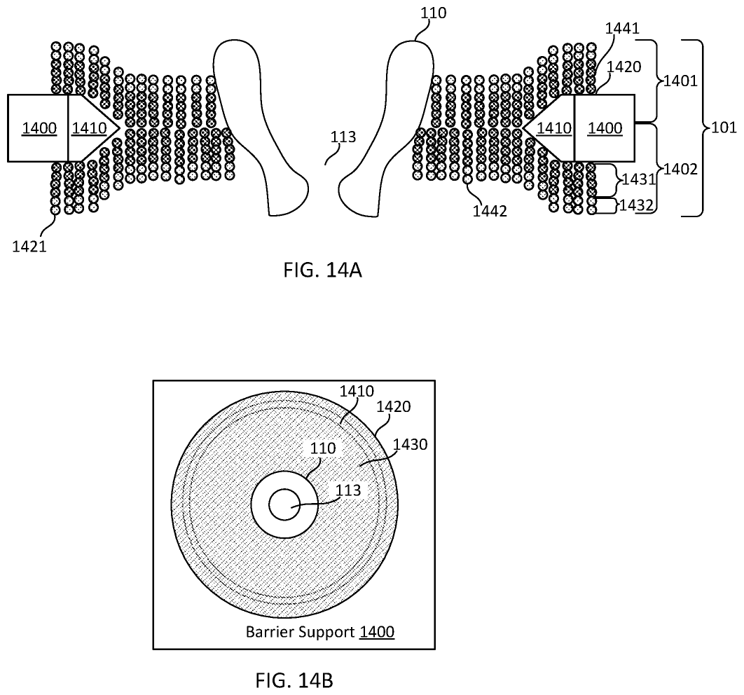

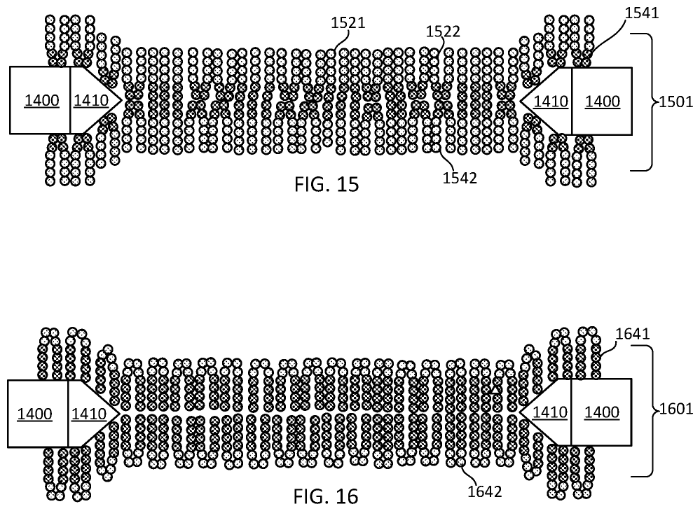

Referring now to FIG. 2B, barrier 201 uses a diblock “AB” copolymer. Barrier 201 includes first layer 207 which may contact fluid 120 and second layer 208 which may contact fluid 120′ in a manner similar to that of layers 107 and 108 described with reference to FIG. 1. First layer 207 includes a first plurality of molecules 202 of a diblock AB copolymer, and second layer 208 includes a second plurality of the molecules 202 of the diblock AB copolymer. As illustrated in FIG. 2B, each molecule 202 of the diblock copolymer includes a hydrophobic block, denoted “B” and being approximately of length “B,” coupled to a hydrophilic block, denoted “A” and being approximately of length “A”. The hydrophilic A blocks of the first plurality of molecules 202 (the molecules forming layer 207) form a first outer surface of the barrier 201, e.g., contact fluid 120. The hydrophilic A blocks of the second plurality of molecules 202 (the molecules forming layer 208) form a second outer surface of the barrier 202, e.g., contact fluid 120′. The respective ends of the hydrophobic B blocks of the first and second pluralities of molecules contact one another within the barrier 201 in a manner such as illustrated in FIG. 2B. As illustrated, substantially all of the molecules 202 within layer 207 may extend substantially linearly and in the same orientation as one another, and similarly substantially all of the molecules 202 within layer 208 may extend substantially linearly and in the same orientation as one another (which is opposite that of the orientation the molecules within layer 207). Accordingly, first and second layers 207, 208 each may have a thickness of approximately A+B, and barrier 201 may have a thickness of approximately 2A+2B. In some examples, length A is about 2 repeating units (RU) to about 100 RU, or about 1 repeating unit (RU) to about 50 RU, e.g., about 5 RU to about 40 RU, or about 10 RU to about 30 RU, or about 10 RU to about 20 RU, or about 20 RU to about 40 RU. Additionally, or alternatively, in some examples, length B is about 2 RU to about 100 RU, 5 RU to about 100 RU, e.g., about 10 RU to about 80 RU, or about 20 RU to about 50 RU, or about 50 RU to about 80 RU. Optionally, barrier 201 described with reference to FIG. 2B may be suspended across an aperture in a manner such as described with reference to FIGS. 14A-14B.

Referring now to FIG. 2C, barrier 211 uses a triblock “BAB” copolymer. Barrier 211 includes first layer 217 which may contact fluid 120 and second layer 218 which may contact fluid 120′ in a manner similar to that of layers 107 and 108 described with reference to FIG. 1. First layer 217 includes a first plurality of molecules 212 of a triblock copolymer, and second layer 218 includes a second plurality of the molecules 212 of the triblock copolymer. As illustrated in FIG. 2C, each molecule 212 of the triblock copolymer includes first and second hydrophobic blocks, each denoted “B” and being approximately of length “B,” and a hydrophilic block disposed between the first and second hydrophobic blocks, denoted “A” and being approximately of length “A”. The hydrophilic A blocks of the first plurality of molecules 212 (the molecules forming layer 217) form a first outer surface of the barrier 211, e.g., contact fluid 120. The hydrophilic A blocks of the second plurality of molecules 212 (the molecules forming layer 218) form a second outer surface of the barrier 211, e.g., contact fluid 120′. The respective ends of the hydrophobic B blocks of the first and second pluralities of molecules contact one another within the barrier 211 in a manner such as illustrated in FIG. 2C. As illustrated, substantially all of the molecules 212 within layer 217 may extend in the same orientation as one another, and may be folded at the A block so that the A block can contact the fluid while the B blocks are interior to the barrier 211. Similarly, substantially all of the molecules 212 within layer 218 may extend in the same orientation as one another (which is opposite that of the orientation the molecules within layer 217), and may be folded at their A blocks so that the A blocks contact the fluid while the B blocks are interior to the barrier 211. Accordingly, first and second layers 217, 218 each may have a thickness of approximately A/2+B, and barrier 211 may have a thickness of approximately A+2B. In some examples, length A is about 2 RU to about 100 RU, e.g., about 10 RU to about 80 RU, or about 20 RU to about 50 RU, or about 50 RU to about 80 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU. Optionally, barrier 211 described with reference to FIG. 2C may be suspended across an aperture in a manner such as described with reference to FIG. 16.

It will be appreciated that the layers of the various barriers provided herein may be configured so as to have any suitable dimensions. Illustratively, to form barriers of similar dimension as one another:

A-B-A triblock copolymer (FIG. 2A) may have 2 hydrophilic blocks, each of length A (each A block is of M_{w}=x) and 1 hydrophobic block of length B (M_{w}=y); when self-assembled, those A-B-A triblock copolymers would form membranes with a top hydrophilic layer of length A, a core hydrophobic layer of length B, and a bottom hydrophilic layer of length A.

A-B diblock copolymer (FIG. 2B) may have 1 hydrophilic block of length A (M_{w}=x), and 1 hydrophobic block of length B (M_{w}=y/2); when self-assembled, those A-B diblock copolymers would form membranes with a top hydrophilic layer of length A, a core hydrophobic layer of length 2B, and a bottom hydrophilic layer of length A.

B-A-B triblock copolymer (FIG. 2C) may have 1 hydrophilic block of length A (M_{w}=x), and 2 hydrophobic blocks, each of length of B (each B block is of M_{w}=y/2); when self-assembled, those B-A-B triblock copolymers would form membranes with a top hydrophilic layer of length A/2, a core hydrophobic layer of length 2B, and a bottom hydrophilic layer of length A/2.

Additionally, or alternatively, the polymer packing into the layer(s) of the membrane may affect the hydrophilic ratio for each of the barriers, where hydrophilic ratio may be defined as the ratio between molecular mass of the hydrophilic block and the total molecular weight (MW or M_{w}) of the block copolymer (BCP) (hydrophilic ratio=M_{w} hydrophilic block/M_{w} BCP). For example:

A-B-A triblock copolymer (FIG. 2A), hydrophilic ratio=2x/(2x+y);

A-B diblock copolymer (FIG. 2B), hydrophilic ratio=x/(x+y/2); and

B-A-B triblock copolymer (FIG. 2C), hydrophilic ratio=x/(x+y).

FIGS. 14A-14B schematically illustrate plan and cross-sectional views of further details of one nonlimiting example of the nanopore composition and device of FIG. 1. More specifically, in the example illustrated in FIGS. 14A-14B, barrier 101 may be suspended using barrier support 1400 defining aperture 1430. For example, barrier support 1400 may include a substrate having an aperture 1430 defined therethrough, e.g., a substantially circular aperture, or an aperture having another shape. Additionally, or alternatively, the barrier support may include one or more features of a well in which the nanopore device is formed, such as a lip or ledge on either side of the well. Nonlimiting examples of materials which may be included in a barrier support are provided further above. An annulus 1410 including hydrophobic (non-polar) solvent, and which also may include other compound(s), may adhere to barrier support 1400 and may support a portion of barrier 101, e.g., may be located within barrier 101 (here, between layer 1401 and layer 1402). Additionally, annulus 1410 may taper inwards in a manner such as illustrated in FIG. 14A. An outer portion of the molecules 1421 of barrier 101 may be disposed on support 1400 (e.g., the portion extending between aperture 1430 and barrier periphery 1420), while an inner portion of the molecules may form a freestanding portion of barrier 101 (e.g., the portion within aperture 1410, a part of which is supported by annulus 1410). Barrier 101 may be prepared, and nanopore 110 may be inserted into the freestanding portion of barrier 101, using operations such as described elsewhere herein. Although FIGS. 14A-14B illustrate nanopore 110 within barrier 101, it should be understood that the nanopore may be omitted, and that barrier 101 may be used for any suitable purpose. More generally, it should be appreciated that while the barriers described herein are particularly suitable for use with nanopores (e.g., for nanopore sequencing such as described with reference to FIGS. 4-7 and 17), the present barriers need not necessarily have nanopores inserted therein.

In the nonlimiting example illustrated in FIG. 14A, barrier 101 may include first layer 1401 including a first plurality of amphiphilic molecules 1421 and second layer 1402 including a second plurality of the amphiphilic molecules contacting the first plurality of amphiphilic molecules. In the nonlimiting example illustrated in FIG. 14A, the copolymer is a diblock copolymer (AB) such as described with reference to FIG. 2B. Here, each molecule 1421 includes a hydrophobic “B” block 1431 (within which circles 1441 with darker fill represent hydrophobic monomers) and a hydrophilic “A” block 1432 (within which circles 1442 with lighter fill represent hydrophilic monomers) coupled directly or indirectly thereto. In other examples such as will be described with reference to FIGS. 15 and 16, the copolymer instead may include a triblock copolymer (e.g., ABA or BAB, respectively). In the example illustrated in FIG. 14A, the hydrophilic blocks 1432 of the first plurality of molecules 1421 may form a first outer surface of barrier 101, e.g., the surface of barrier 101 contacting fluid 120 on first side 111. The hydrophilic blocks 1432 of the second plurality of molecules 1421 may form a second outer surface of barrier 101, e.g., the surface of barrier 101 contacting fluid 120′ on second side 112. The hydrophobic blocks 1431 of the first and second pluralities of molecules 1421 may contact one another within the barrier.

Although FIGS. 14A-14B illustrate a suspended barrier that includes a diblock copolymer, it will be appreciated that suspended barriers that included other types of polymers provided herein, are similarly contemplated. FIG. 15 schematically illustrates an alternative barrier that may be used in the example described with reference to FIGS. 14A-14B. FIG. 15 illustrates barrier 1501 which is suspended using barrier support 1400 and annulus 1410 in a manner such as described with reference to FIGS. 14A-14B. In this example, barrier 1501 includes molecules of an ABA triblock copolymer such as described with reference to FIG. 2A. Here, the triblock copolymer includes hydrophobic “B” sections 1541 coupled to and between hydrophilic “A” sections 1542. In the example shown in FIG. 15, each individual ABA molecule may be in one of two arrangements. For example, ABA molecules 1521 may extend through the layer in a linear fashion, with an “A” section on each side of the barrier and the “B” section in the middle of the barrier. Or, for example, ABA molecules 1522 may extend to the middle of the barrier and then fold back on themselves, so that both “A” sections are on the same side of the barrier and the “B” section is in the middle of the barrier. Accordingly, in this example, barrier 1501 may be considered to be partially a single layer and partially a bilayer. In other examples (not specifically illustrated) in which barrier 1501 substantially includes molecules 1521 which extend through the barrier in linear fashion, barrier 1501 may substantially be a monolayer. In still other examples (not specifically illustrated) in which barrier 1501 substantially includes molecules 1522 which extend to approximately the middle of the barrier and then fold back on themselves, barrier 1501 may substantially be a bilayer. A nanopore, not specifically, shown, optionally may be inserted into any of such options for barrier 1501 in a manner similar to that described elsewhere herein, e.g., as illustrated in FIGS. 14A-14B.

FIG. 16 schematically illustrates an alternative barrier that may be used in the example described with reference to FIGS. 14A-14B. FIG. 16 illustrates barrier 1601 which is suspended using barrier support 1400 and annulus 1410 in a manner such as described with reference to FIGS. 14A-14B. In this example, barrier 1601 includes molecules of a BAB triblock copolymer such as described with reference to FIG. 2C. Here, the triblock copolymer includes hydrophilic “A” sections 1642 coupled to and between hydrophobic “B” sections 1641. In this example, barrier 1601 may have a bilayer architecture with the “B” sections 1641 oriented towards each other. The hydrophobic ends of the BAB molecules generally may located approximately in the middle of barrier 1601, the molecules then extend towards either outer surface of the barrier, and then fold back on themselves. As such, both “B” sections are located in the middle of the barrier and the “A” section is on one side or the other of the barrier. A nanopore, not specifically, shown, optionally may be inserted into barrier 1601 in a manner similar to that described elsewhere herein, e.g., as illustrated in FIGS. 14A-14B.

The present diblock and triblock copolymers may include any suitable combination of hydrophobic and hydrophilic blocks. In some examples, the hydrophilic A block may include a polymer selected from the group consisting of: N-vinyl pyrrolidone, polyacrylamide, zwitterionic polymer, hydrophilic polypeptide, nitrogen containing units, and poly(ethylene oxide) (PEO). Illustratively, the polyacrylamide may be selected from the group consisting of: poly(N-isopropyl acrylamide) (PNIPAM), and charged polyacrylamide, and phosphoric acid functionalized polyacrylamide. Nonlimiting examples of zwitterionic monomers that may be polymerized to form zwitterionic polymers include:

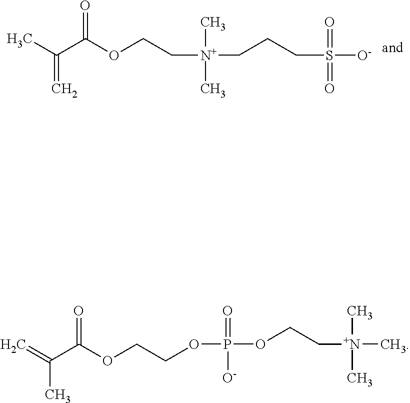 [SMILES: C=C(C)C(=O)OCC[N+](C)(C)CCCS(=O)(=O)[O-] ; C=C(C)C(=O)OCCOP(=O)([O-])OCC[N+](C)(C)C]

Nonlimiting examples of hydrophilic polypeptides include:

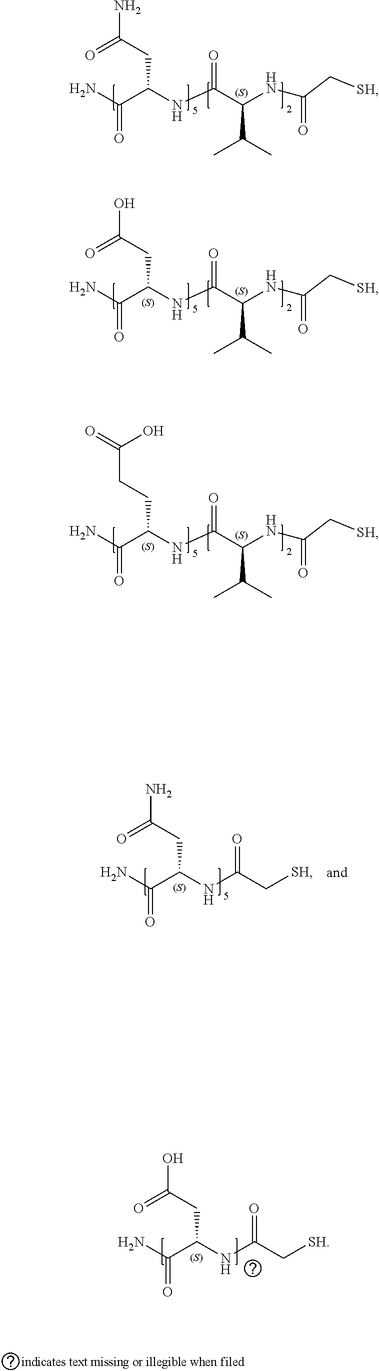

A nonlimiting example of a charged polyacrylamide is

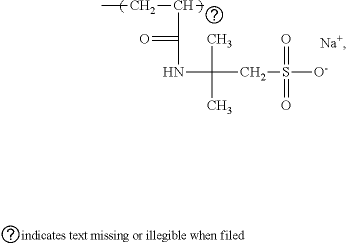

where n is between about 2 and about 100. Nonlimiting examples of nitrogen containing units include:

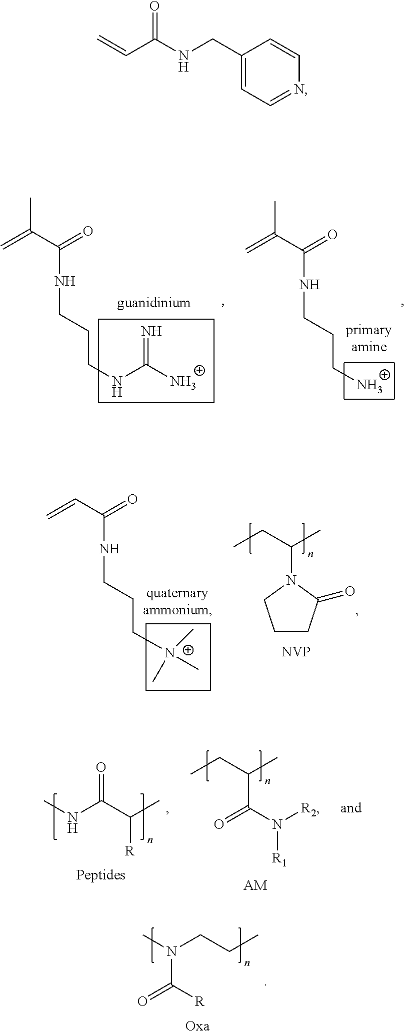

In some examples, the hydrophobic B block may include a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB). Nonlimiting examples of hydrogenated polydienes include saturated polybutadiene (PBu), saturated polyisoprene (PI), saturated poly(myrcene),

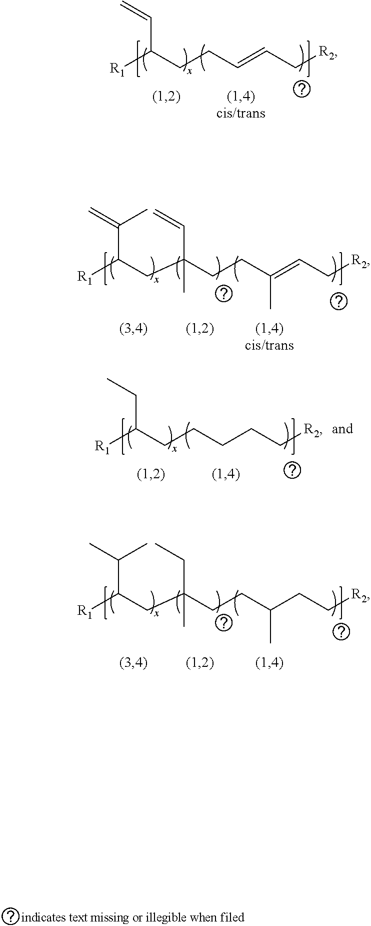

where n is between about 2 and about 100, x is between about 2 and about 100, y is between about 2 and about 100, z is between about 2 and about 100, R₁ is a functional group selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen, and R₂ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. In other nonlimiting examples of hydrogenated polydienes, R₁ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. A nonlimiting example of fluorinated polyethylene is

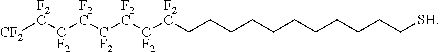

Nonlimiting examples of hydrophobic polypeptides include (0<x<1):

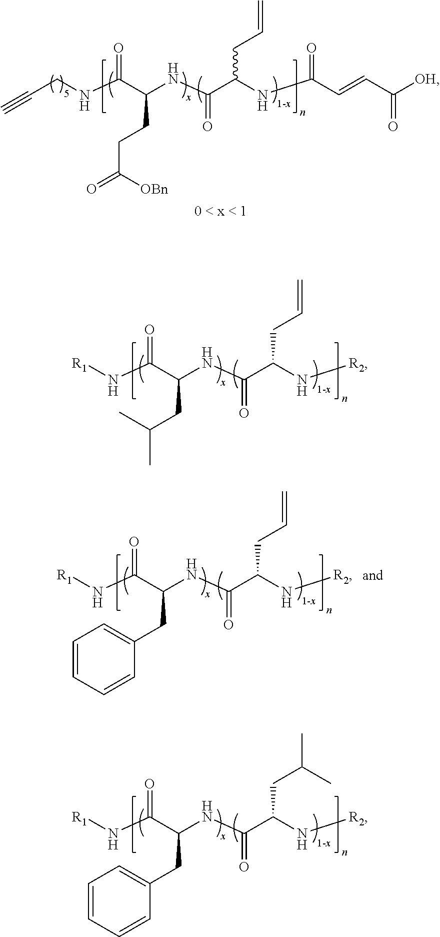

where n is between about 2 and about 100.

In one nonlimiting example, an AB diblock copolymer includes PDMS-b-PEO, where “-b-” denotes that the polymer is a block copolymer. In another nonlimiting example, an AB diblock copolymer includes PBd-b-PEO. In another nonlimiting example, an AB diblock copolymer includes PIB-b-PEO. In another nonlimiting example, a BAB triblock copolymer includes PDMS-b-PEO-b-PDMS. In another nonlimiting example, a BAB triblock copolymer includes PBd-b-PEO-b-PBd. In another nonlimiting example, a BAB triblock copolymer includes PIB-b-PEO-b-PIB. In another nonlimiting example, an ABA triblock copolymer includes PEO-b-PBd-b-PEO. In another nonlimiting example, an ABA triblock copolymer includes PEO-b-PDMS-b-PEO. In another nonlimiting example, an ABA triblock copolymer includes PEO-b-PIB-b-PEO. It will be appreciated that any suitable hydrophilic block(s) may be used with any suitable hydrophobic block(s). Additionally, in examples including two hydrophilic blocks, those blocks may be but need not necessarily include the same polymers as one another. Similarly, in examples including two hydrophobic blocks, those blocks may be, but need not necessarily include, the same polymers as one another.

The respective molecular weights, glass transition temperatures, and chemical structures of the hydrophobic and hydrophilic blocks suitably may be selected so as to provide the barrier with appropriate stability for use and ability to insert a nanopore. For example, the respective molecular weights of the hydrophobic and hydrophilic blocks may affect how thick each of the blocks (and thus layers of the barrier) are, and may influence stability as well as capacity to insert the nanopore, e.g., through electroporation, pipette pump cycle, or detergent assisted pore insertion. Additionally, or alternatively, the ratio of molecular weights of the hydrophilic and hydrophobic blocks may affect self-assembly of those blocks into the layers of the barrier. Additionally, or alternatively, the respective glass transition temperatures (T_{g}) of the hydrophobic and hydrophilic blocks may affect the lateral fluidity of the layers of the barrier; as such, in some examples it may be useful for the hydrophobic and/or hydrophilic blocks to have a T_{g} of less than the operating temperature of the device, e.g., less than room temperature, and in some examples less than about 0° C. Additionally, or alternatively, chemical structures of the hydrophobic and hydrophilic blocks may affect the way the chains get packed into the layers, and stability of those layers.

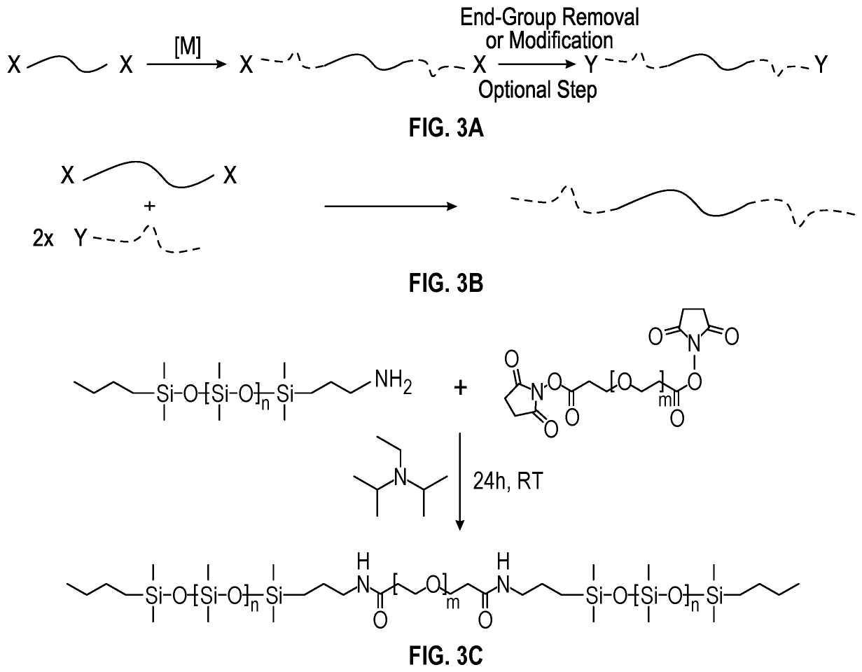

It will be appreciated that the present diblock and triblock copolymers may be made using any suitable combination of operations. FIGS. 3A-3C schematically illustrate example schemes for preparing triblock copolymers for use in the nanopore composition and device of FIG. 1. In some examples, the present diblock and triblock copolymers may be made using a “macro-initiator” approach such as illustrated in FIG. 3A in which one polymer block is made first and then used as an initiator (X in FIG. 3A) to grow one or more additional blocks using monomers ([M] in FIG. 3A). Illustratively, operations for making a diblock copolymer may include polymerizing a plurality of hydrophilic monomers to form a hydrophilic polymer; forming an initiator at a terminal end of the hydrophilic polymer; and using the initiator to polymerize a plurality of hydrophobic monomers to form a hydrophobic polymer coupled to the hydrophilic polymer. Alternatively, operations for making a diblock copolymer may include polymerizing a plurality of hydrophobic monomers to form a hydrophobic polymer; forming an initiator at a terminal end of the hydrophobic polymer; and using the initiator to polymerize a plurality of hydrophilic monomers to form a hydrophilic polymer coupled to the hydrophobic polymer. Similarly, operations for making a triblock BAB copolymer may include polymerizing a plurality of hydrophilic monomers to form a hydrophilic polymer; forming initiators at respective terminal ends of the hydrophilic polymer; and using the initiators to polymerize a plurality of hydrophobic monomers to form a hydrophobic polymer coupled to each terminal end of the hydrophilic polymer. Similarly, operations for making a triblock ABA copolymer may include polymerizing a plurality of hydrophobic monomers to form a hydrophobic polymer; forming initiators at respective terminal ends of the hydrophobic polymer; and using the initiators to polymerize a plurality of hydrophilic monomers to form a hydrophilic polymer coupled to each terminal end of the hydrophobic polymer. In such a “macro-initiator” approach, the initiator (X in FIG. 3A) suitably may be selected based on the particular monomers being used and the particular type of polymerization being performed. For example, for an atom transfer free radical polymerization (ATRP), the initiator may include bromine or chlorine. Or, for example, for a reversible addition fragmentation chain transfer (RAFT) polymerization, the initiator may include a chain transfer agent. After polymerization is complete, the end group(s) (X in FIG. 3A) may be modified or removed (e.g., to provide end group(s) Y in FIG. 3A).

In other examples, the present diblock and triblock copolymers may be made using a “coupling” approach such as illustrated in FIG. 3B in which polymer blocks are made separately and then coupled together using reactive moieties (X and Y in FIG. 3B). Illustratively, operations for making a diblock copolymer may include polymerizing a plurality of hydrophilic monomers to form a hydrophilic polymer; polymerizing a plurality of hydrophobic monomers to form a hydrophobic polymer; and coupling the hydrophilic polymer to the hydrophobic polymer. Operations for making a triblock copolymer may include polymerizing a plurality of hydrophilic monomers to form a hydrophilic polymer having terminal ends; polymerizing a plurality of hydrophobic monomers to form first and second hydrophobic polymers; and coupling the first and second hydrophobic polymers to respective terminal ends of the hydrophilic polymer. Alternatively, operations for making a triblock copolymer may include polymerizing a plurality of hydrophilic monomers to form first and second hydrophilic polymers; polymerizing a plurality of hydrophobic monomers to form a hydrophobic polymer having terminal ends; and coupling the first and second hydrophilic polymers to respective terminal ends of the hydrophobic polymer. In such a “coupling” approach, a terminal end of the hydrophobic polymer may include a first reactive moiety (Y in FIG. 3B), and a terminal end of the hydrophilic polymer may include a second reactive moiety (X in FIG. 3B) that reacts with the first reactive moiety to couple the hydrophilic polymer to the hydrophobic polymer. The reactive moieties (X and Y in FIG. 3B) suitably may be selected based on the particular polymers being coupled and the type of coupling being performed. For example, “Click” chemistry moieties may be used. Illustratively, one of the first and second reactive moieties may include an azide and the other of the first and second reactive moieties may include an alkyne; or one of the first and second reactive moieties may include a thiol and the other of the first and second reactive moieties may include an alkene; or one of the first and second reactive moieties may include a thiol and the other of the first and second reactive moieties comprises an alkyne. Or, for example, amide linkers may be formed. Illustratively, one of the first and second reactive moieties may include an amine and the other of the first and second reactive moieties may include N-hydroxysuccinimide (NHS). FIG. 3C illustrates a nonlimiting example in which the hydrophobic polymer is PDMS having an amine (NH₂) group at one of its terminal ends, the hydrophilic polymer is PEO having NHS at its terminal end, and the amine and NHS groups are reacted with one another in the presence of triisopropylamine to provide an ABA triblock copolymer. Another nonlimiting example of forming a triblock copolymer is shown below. In this example, a PDMS-bis allyl is reacted by thiol-ene click chemistry with a PEG-thiol; for the reaction to proceed, it is carried out under inert atmosphere using degassed solvent (e.g., chloroform) and in the presence of a photoinitiator (e.g., irgacure 2959), and under UV exposure for 5-30 min.

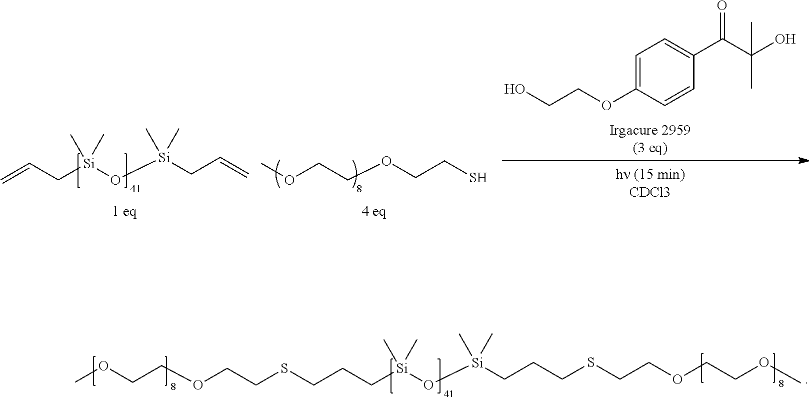

Another nonlimiting example of forming a triblock copolymer is shown below. In this example, a PIB-bis allyl is reacted by thiol-ene click chemistry with a PEG-thiol; for the reaction to proceed, it is carried out under inert atmosphere (dry Argon or dry Nitrogen) using degassed solvent (e.g., chloroform) and in the presence of a photoinitiator (e.g., irgacure 2959), and under UV exposure for 5-60 min. In some examples, the reaction is carried out under UV exposure for 2-180 min. with a UV power ranging from 1 mW/cm² to 100 mW/cm². In some examples, the UV wavelength used is 365 nm.

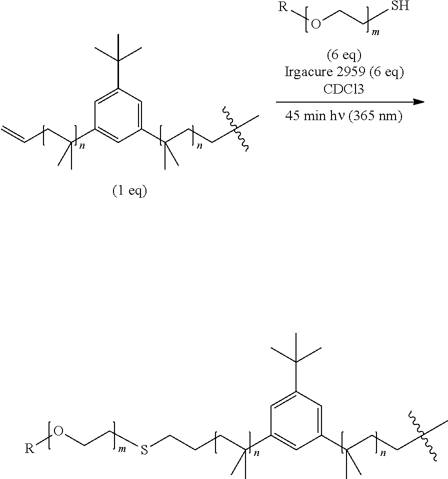

For nanopore sequencing applications, membrane fluidity can be considered beneficial. Without wishing to be bound by any theory, the fluidity of a block copolymer membrane is believed to be largely imparted by the physical property of the hydrophobic “B” blocks. More specifically, B blocks including “low T_{g}” hydrophobic polymers (e.g., having a T_{g} below around 0° C.) may be used to generate membranes that are more fluid than those with B blocks including “high T_{g}” polymers (e.g., having a T_{g} above room temperature). For example, in certain examples, a hydrophobic B block of the copolymer has a T_{g} of less than about 20° C., less than about 0° C., or less than about −20° C.

Hydrophobic B blocks with a low T_{g} may be used to help maintain membrane flexibility under conditions suitable for performing nanopore sequencing, e.g., in a manner such as described with reference to FIG. 4, 5, 6, 7, or 17. In some examples, hydrophobic B blocks with a sufficiently low T_{g} for use in nanopore sequencing may include, or may consist essentially of, PIB, which may be expected to have a T_{g} in the range of about −75° C. to about −25° C. In other examples, hydrophobic B blocks with a sufficiently low T_{g} for use in nanopore sequencing may include, or may consist essentially of, PDMS, which may be expected to have a T_{g} in the range of about −135° C. (or lower) to about −115° C. In still other examples, hydrophobic B blocks with a sufficiently low T_{g} for use in nanopore sequencing may include, or may consist essentially of, PBd. Different forms of PBd may be used as B blocks in the present barriers. For example, the cis-1,4 form of PBd may be expected to have a T_{g} in the range of about −105° C. to about −85° C. Or, for example, the cis-1,2 form of PBd may be expected to have a T_{g} in the range of about −25° C. to about 0° C. Or, for example, the trans-1,4 form of PBd may be expected to have a T_{g} in the range of about −95° C. to about −5° C. In yet other examples, hydrophobic B blocks with a sufficiently low T_{g} for use in nanopore sequencing may include, or may consist essentially of, polymyrcene (PMyr), which may be expected to have a T_{g} in the range of about −75° C. to about −45° C. In yet other examples, hydrophobic B blocks with a sufficiently low T_{g} for use in nanopore sequencing may include, or may consist essentially of, polyisoprene (PIP). Different forms of PIP may be used as B blocks in the present barriers. For example, the cis-1,4 form of PIP may be expected to have a T_{g} in the range of about −85° C. to about −55° C. Or, for example, the trans-1,4 form of PIP may be expected to have a T_{g} in the range of about −75° C. to about −45° C.

Hydrophobic B blocks with a fully saturated carbon backbone, such as PIB, also may be expected to increase chemical stability of the block copolymer membrane. Additionally, or alternatively, branched structures within the hydrophobic B block, such as with PIB, may be expected to induce chain entanglement, which may be expected to enhance the stability of the block copolymer membrane. This may allow for a smaller hydrophobic block to be used, ameliorating the penalty of hydrophobic mismatch towards an inserted nanopore. Additionally, or alternatively, hydrophobic B blocks with relatively low polarity may be expected to be better electrical insulators, thus improving electrical performance of a device for nanopore sequencing (e.g., such as described with reference to FIG. 4-7 or 17).

In some examples of the AB copolymer shown below including PBd as the B block and PEO as the A block, R is a functional group selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen; m=about 2 to about 100; and n=about 2 to about 100.

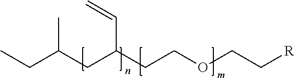

In some nonlimiting examples, R=OH; n=about 8 to about 50; and m=about 1 to about 20. In some nonlimiting examples, R=OH; n=about 10 to about 15; and m=about 5 to about 15.

In some examples of the ABA copolymer shown below including one or more PIB blocks as the B block and PEO as the A block, R₁ and R₂ are independently moieties selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen; V is an optional group that corresponds to a bis-functional initiator from which the isobutylene may be propagated and can be tert-butylbenzene, a phenyl connected to the hydrophobic blocks via the para, meta, or ortho positions, naphthalene, another aromatic group, an alkane chain with between about 2 and about 20 carbons, or another aliphatic group; m=about 2 to about 100; and n=about 2 to about 100. V may optionally be flanked by functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen. When V is absent, only one PIB block is present and n=about 2 to about 100. L₁ and L₂ are independently linkers, which may include at least one moiety selected from the group consisting of an amide, a thioether (sulfide), a succinic group, a maleic group, a methylene, an ether, and a product of a click reaction.

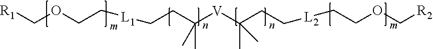

In some nonlimiting examples of the above structure, n=about 2 to about 50, and m=about 1 to about 50, R₁=R₂=COOH, V=tert-butylbenzene, and L₁=L₂=ethyl sulfide. In other nonlimiting examples, n=about 5 to about 20, m=about 2 to about 15, R₁=R₂=COOH, V=tert-butylbenzene, and L₁=L₂=ethyl sulfide. In other nonlimiting examples, n=about 13 to about 19, m=about 2 to about 5, R₁=R₂=COOH, V=tert-butylbenzene, and L₁=L₂=ethyl sulfide. In other nonlimiting examples, n=about 7 to about 13, m=about 7 to about 13, R₁=R₂=COOH, V=tert-butylbenzene, and L₁=L₂=ethyl sulfide. In particular, in one nonlimiting example (the structure of which is shown below), n=16, m=3, R₁=R₂=COOH, V=tert-butylbenzene, and L₁=L₂=ethyl sulfide. In another nonlimiting example (the structure of which is shown below), n=10, m=10, and R₁=R₂=COOH, V=tert-butylbenzene, and L₁=L₂=ethyl sulfide. In another nonlimiting example (the structure of which is shown below), n=16, m=8, R₁=R₂=CH₃, V=tert-butylbenzene, and L₁=L₂=ethyl sulfide.

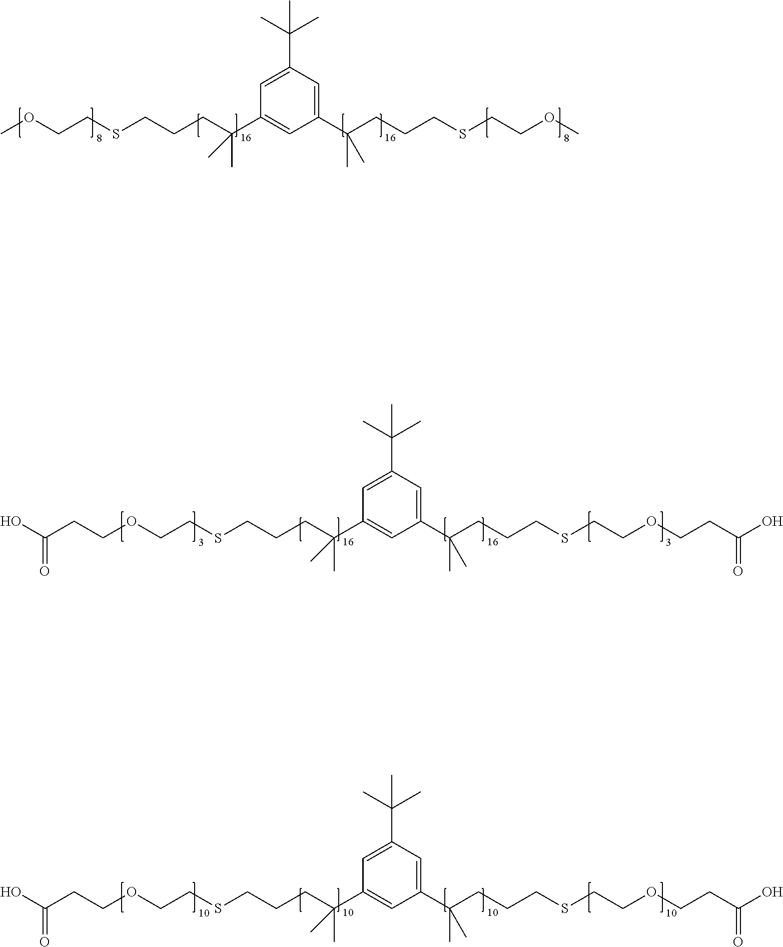

In some examples, multifunctional precursors may be sourced and used as precursors to the synthesis of bifunctional initiators to which V corresponds in the example further above. For example, the multifunctional precursor may be 5-tert-butylisophthalic acid (TBIPA) which can be synthesized into 1-(tert-butyl)-3,5-bis(2-methoxypropan-2-yl)benzene (TBDMPB) using reactions known in the art. In another example, TBIPA may be synthesized into 1-tert-butyl-3,5-bis(2-chloropropan-2-yl)benzene using reactions known in the art. The use of such bifunctional initiators allows cationic polymerization on both sides of the initiator, generating bifunctional PIBs, such as allyl-PIB-allyl, which can then be coupled to hydrophilic A blocks to generate ABA block copolymers including PIB as the B block. Here, although the bifunctional initiator may be located between first and second PIB polymers, it should be understood that the first and second PIB polymers and the bifunctional initiator (V) together may be considered to form a B block, e.g., of an ABA triblock copolymer.

In another nonlimiting example, an ABA triblock copolymer includes

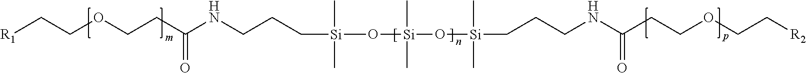

where m=about 2 to about 100, n=about 2 to about 100, p=about 2 to about 100, R₁ and R₂ are independently functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen. In some nonlimiting examples, m=about 2 to about 30, n=about 25 to about 45, p=about 2 to about 30, R₁ and R₂ are independently functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen. In some nonlimiting examples, m=about 2 to about 15, n=about 30 to about 40, p=about 2 to about 15, R₁ and R₂ are independently functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen. In some nonlimiting examples, m=about 7 to about 11, n=about 35 to about 40, p=about 7 to about 11, R₁ and R₂ are independently functional groups selected from the group consisting of a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen. In some nonlimiting examples, m=about 2 to about 5, n=about 30 to about 37, p=about 2 to about 5, R₁ and R₂ are independently functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen.

In particular, as shown below, in one nonlimiting example, m=3, n=34, p=3, and R₁=R₂=COOH. In another nonlimiting example shown below, m=9, n=37, p=9, and R₁=R₂=COOH.

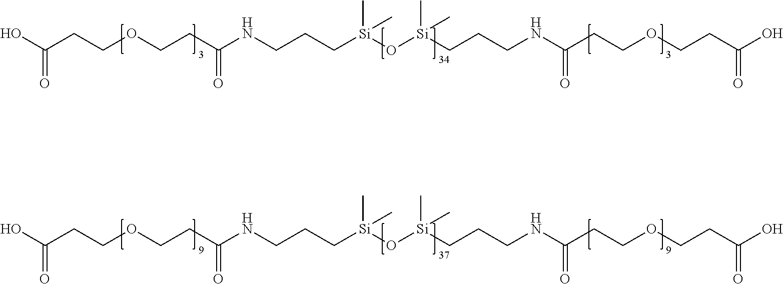

In some examples of the AB copolymer shown below including a PIB block as the B block and PEO as the A block, R is a moiety selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen; m=about 2 to about 100; n=about 2 to about 100; and L is a linker selected from the group consisting of an amide, a thioether (sulfide), a succinic group, a maleic group, a methylene, an ether, or a product of a click reaction.

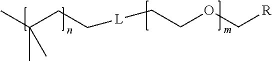

In particular, as shown below, in one nonlimiting example, n=13, m=8, R is methyl, and L is ethyl sulfide. In another nonlimiting example shown below, n=13, m=3, R is a carboxyl group, and L is ethyl sulfide. In another nonlimiting example shown below, n=30, m=8, R is methyl, and L is ethyl sulfide. In another nonlimiting example shown below, n=30, m=3, R is a carboxyl group, and L is ethyl sulfide.

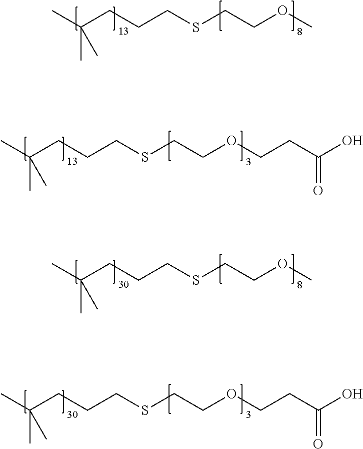

It will be appreciated that nanopore devices such as described with reference to FIG. 1 may be made using any suitable barriers such as, but not limited to, those described with reference to FIGS. 2A-2C. Additionally, the barriers may be made using any suitable copolymers such as, but not limited to, those described with reference to FIGS. 3A-3C. For example, device 100 described with reference to FIG. 1 may be made using operations that include forming the barrier in the fluidic well; and inserting the nanopore within the barrier. The barrier may be suspended using a barrier support 1400 in a manner such as described with reference to FIGS. 14A-14B, 15, and 16. Forming the barrier may include “painting” as known in the art. Known techniques for painting barriers that are suspended by barrier supports include brush painting (manual), mechanical painting (e.g., using stirring bar), and bubble painting (e.g., using flow through the device). Known techniques for inserting a nanopore into a suspended barrier include electroporation, pipette pump cycle, and detergent assisted pore insertion. Tools for forming suspended barriers using synthetic polymers and inserting nanopores in the suspended barriers are commercially available, such as the Orbit 16 TC platform available from Nanion Technologies Inc. (California, USA).

In some examples, the barrier may have a survival rate of 70% or more, 80% or more, 90% or more, or 95% or more when subjected to a voltage of 450 mV across the barrier. In some examples, the barrier has an open pore current at 100 mV of 95 pA or more, or 100 pA or more. In some examples, the barrier has an open pore current at 50 mV of 32 pA or more, 34 pA or more, or 36 pA or more. In some examples, the barrier-pore RMS noise is 2.2 pA or less, 2.0 pA or less, 1.8 pA or less, 1.6 pA or less, or 1.5 pA or less. In some examples, the barrier has a signal-to-noise ratio of 40 or more, 50 or more, 60 or more, or 70 or more. In some examples, the barrier has a membrane painting yield of 90% or more, or 95% or more.

In some examples, the barrier has a pore insertion voltage between about 300 mV and about 1100 mV. In some examples, the barrier has a single pore percentage after insertion of 85% or more, 90% or more, or 95% or more. In some examples, the barrier has a single pore survival rate of 90% or more, or 95% or more. In some examples, the barrier has a single pore current standard deviation of 2 pA or less, 1 pA or less, or 0.5 pA or less.

In some examples, a waveform is applied to the barrier which is made of a train of positive voltage micro pulses, spaced by negative voltage periods at −100 mV for 100 ms. The train of positive voltage pulses has a total of 20 pulses, with duration of 10 μs. The spacings between them have a set duration value of 30 ms and a voltage held at +50 mV. During a first cycle, the waveform may be applied continuously for a period of 5 minutes and the magnitude of the pulses kept at +700 mV. In further applied cycles (applied again for 5 mins each), the pulsing intensity is increased from +700 mV to +1200 mV in 100 mV steps, for a total of six cycles. In some examples, the membrane survival rate under such a waveform is 60% or more, 80% or more, 90% or more, or 95% or more. In some examples, the voltage at 50% membrane survival is 1000 mV or more, or 1200 mV or pore. In some examples, the voltage at 50% membrane and single pore survival is 900 mV or more, or 1000 mV or more.

It will further be appreciated that the present barriers may be used in any suitable device or application. For example, FIG. 4 schematically illustrates a cross-sectional view of an example use of the composition and device of FIG. 1. Device 100 illustrated in FIG. 4 may be configured to include fluidic well 100′, barrier 101 which may have a configuration such as described with reference to FIGS. 2A-2C, 14A-14B, 15, and/or 16 (that is, barrier 101 optionally may be suspended using a barrier support, and may include any AB, ABA, or BAB copolymer provided herein), first and second fluids 120, 120′, and nanopore 110 in a manner such as described with reference to FIG. 1. In the nonlimiting example illustrated in FIG. 4, second fluid 120′ optionally may include a plurality of each of nucleotides 121, 122, 123, 124, e.g., G, T, A, and C, respectively. Each of the nucleotides 121, 122, 123, 124 in second fluid 120′optionally may be coupled to a respective label 131, 132, 133, 134 coupled to the nucleotide via an elongated body (elongated body not specifically labeled). Optionally, device 100 further may include polymerase 105. As illustrated in FIG. 4, polymerase 105 may be within the second composition of second fluid 120′. Alternatively, polymerase 105 may be coupled to nanopore 110 or to barrier 101, e.g., via a suitable elongated body (not specifically illustrated). Device 100 optionally further may include first and second polynucleotides 140, 150 in a manner such as illustrated in FIG. 4. Polymerase 105 may be for sequentially adding nucleotides of the plurality to the first polynucleotide 140 using a sequence of the second polynucleotide 150. For example, at the particular time illustrated in FIG. 4, polymerase 105 incorporates nucleotide 122 (T) into first polynucleotide 140, which is hybridized to second polynucleotide 150 to form a duplex. At other times (not specifically illustrated), polymerase 105 sequentially may incorporate other of nucleotides 121, 122, 123, 124 into first polynucleotide 140 using the sequence of second polynucleotide 150.

Circuitry 180 illustrated in FIG. 4 may be configured to detect changes in an electrical characteristic of the aperture responsive to the polymerase sequentially adding nucleotides of the plurality to the first polynucleotide 140 using a sequence of the second polynucleotide 150. In the nonlimiting example illustrated in FIG. 4, nanopore 110 may be coupled to permanent tether 410 which may include head region 411, tail region 412, elongated body 413, reporter region 414 (e.g., an abasic nucleotide), and moiety 415. Head region 411 of tether 410 is coupled to nanopore 110 via any suitable chemical bond, protein-protein interaction, or any other suitable attachment that is normally irreversible. Head region 411 can be attached to any suitable portion of nanopore 110 that places reporter region 414 within aperture 413 and places moiety 415 sufficiently close to polymerase 105 so as to interact with respective labels 131, 132, 133, 134 of nucleotides 121, 122, 123, 124 that are acted upon by polymerase 105. Moiety 415 respectively may interact with labels 131, 132, 133, 134 in such a manner as to move reporter region 414 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to nanopores to sequence polynucleotides, see U.S. Pat. No. 9,708,655, the entire contents of which are incorporated by reference herein.

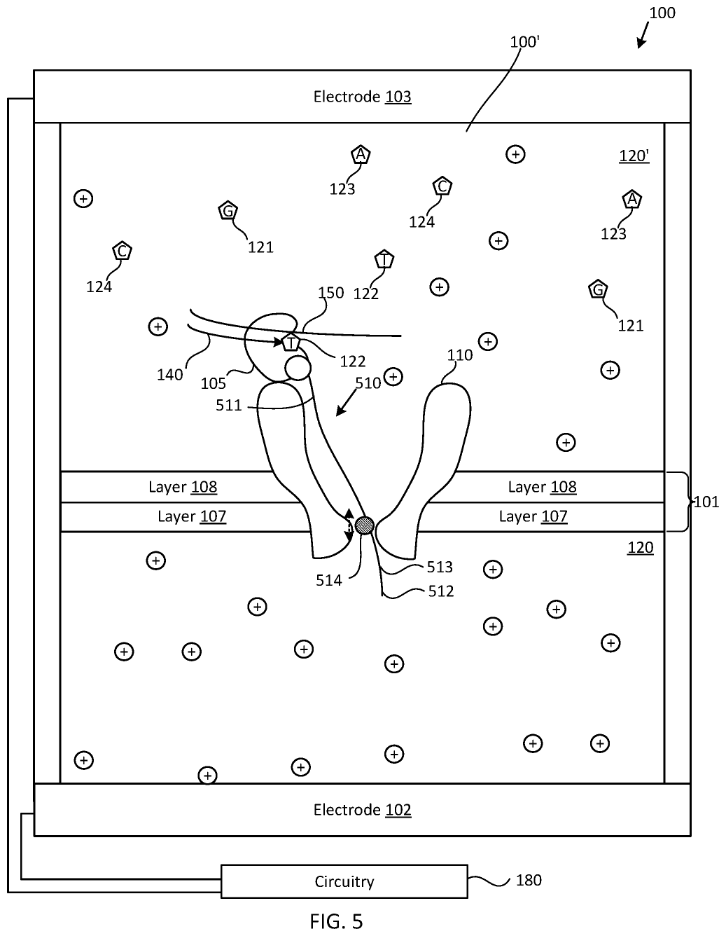

FIG. 5 schematically illustrates a cross-sectional view of another example use of the composition and device of FIG. 1. As illustrated in FIG. 5, device 100 may include fluidic well 100′, barrier 101 which may have a configuration such as described with reference to FIGS. 2A-2C, 14A-14B, 15, and/or 16 (that is, barrier 101 optionally may be suspended using a barrier support, and may include any AB, ABA, or BAB copolymer provided herein), first and second fluids 120, 120′, nanopore 110, and first and second polynucleotides 140, 150, all of which may be configured similarly as described with reference to FIG. 4. In the nonlimiting example illustrated in FIG. 5, nucleotides 121, 122, 123, 124 need not necessarily be coupled to respective labels. Polymerase 105 may be coupled to nanopore 110 and may be coupled to permanent tether 510 which may include head region 511, tail region 512, elongated body 513, and reporter region 514 (e.g., an abasic nucleotide). Head region 511 of tether 510 is coupled to polymerase 105 via any suitable chemical bond, protein-protein interaction, or any other suitable attachment that is normally irreversible. Head region 511 can be attached to any suitable portion of polymerase 105 that places reporter region 514 within aperture 113. As polymerase 105 interacts with nucleotides 121, 122, 123, 124, such interactions may cause polymerase 105 to undergo conformational changes. Such conformational changes may move reporter region 514 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to polymerases to sequence polynucleotides, see U.S. Pat. No. 9,708,655, the entire contents of which are incorporated by reference herein.

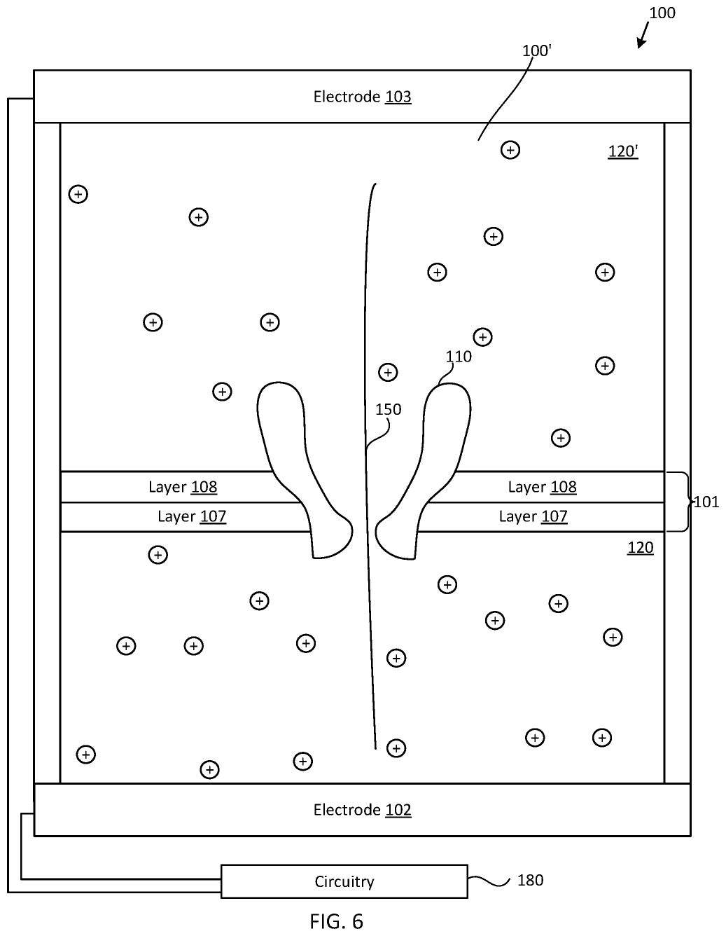

FIG. 6 schematically illustrates a cross-sectional view of another example use of the composition and device of FIG. 1. As illustrated in FIG. 6, device 100 may include fluidic well 100′, barrier 101 which may have a configuration such as described with reference to FIGS. 2A-2C, 14A-14B, 15, and/or 16 (that is, barrier 101 optionally may be suspended using a barrier support, and may include any AB, ABA, or BAB copolymer provided herein), first and second fluids 120, 120′, and nanopore 110 all of which may be configured similarly as described with reference to FIG. 4. In the nonlimiting example illustrated in FIG. 6, polynucleotide 150 is translocated through nanopore 110 under an applied force, e.g., a bias voltage that circuitry 180 applies between electrode 102 and electrode 103. As bases in polynucleotide 150 pass through nanopore 110, such bases may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see U.S. Pat. No. 5,795,782, the entire contents of which are incorporated by reference herein.

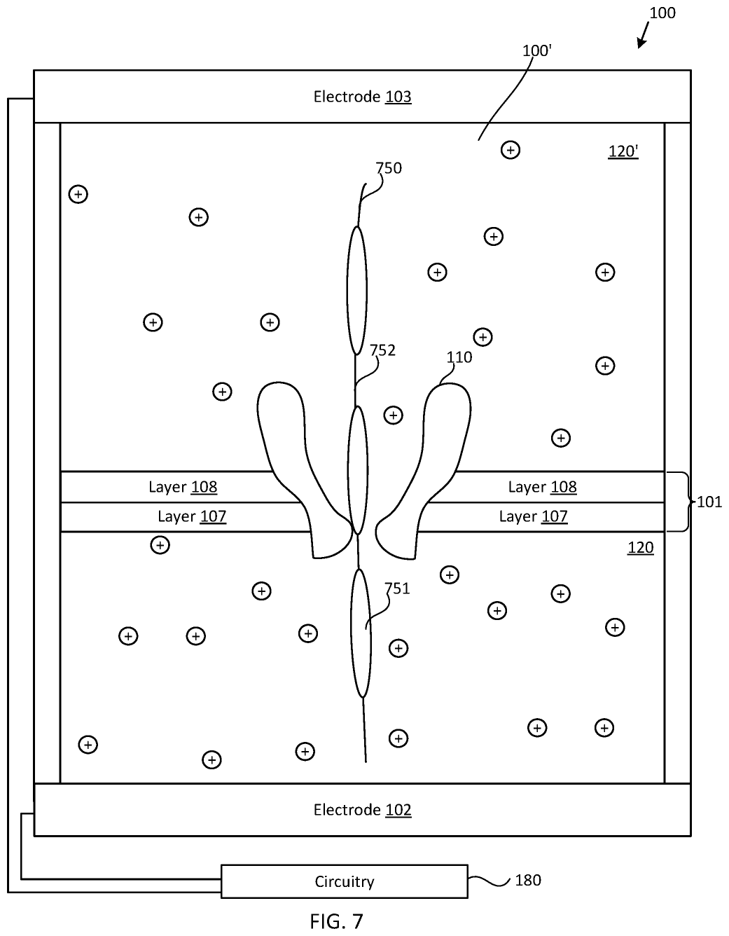

FIG. 7 schematically illustrates a cross-sectional view of another example use of the composition and device of FIG. 1. As illustrated in FIG. 7, device 100 may include fluidic well 100′, barrier 101 which may have a configuration such as described with reference to FIGS. 2A-2C, 14A-14B, 15, and/or 16 (that is, barrier 101 optionally may be suspended using a barrier support, and may include any AB, ABA, or BAB copolymer provided herein), first and second fluids 120, 120′, and nanopore 110 all of which may be configured similarly as described with reference to FIG. 4. In the nonlimiting example illustrated in FIG. 7, surrogate polymer 750 is translocated through nanopore 110 under an applied force, e.g., a bias voltage that circuitry 180 applies between electrode 102 and electrode 103. As used herein, a “surrogate polymer” is intended to mean an elongated chain of labels having a sequence corresponding to a sequence of nucleotides in a polynucleotide. In the example illustrated in FIG. 7, surrogate polymer 750 includes labels 751 coupled to one another via linkers 752. An XPANDOMER™ is a particular type of surrogate polymer developed by Roche Sequencing, Inc. (Pleasanton, Calif.). XPANDOMERS™ may be prepared using Sequencing By eXpansion™ (SBX™, Roche Sequencing, Pleasanton Calif.). In Sequencing by eXpansion™, an engineered polymerase polymerizes xNTPs which include nucleobases coupled to labels via linkers, using the sequence of a target polynucleotide. The polymerized nucleotides are then processed to generate an elongated chain of the labels, separated from one another by linkers which are coupled between the labels, and having a sequence that is complementary to that of the target polynucleotide. For example descriptions of XPANDOMERS™, linkers (tethers), labels, engineered polymerases, and methods for SBX™, see the following patents, the entire contents of each of which are incorporated by reference herein: U.S. Pat. Nos. 7,939,249, 8,324,360, 8,349,565, 8,586,301, 8,592,182, 9,670,526, 9,771,614, 9,920,386, 10,301,345, 10,457,979, 10,676,782, 10,745,685, 10,774,105, and 10,851,405.

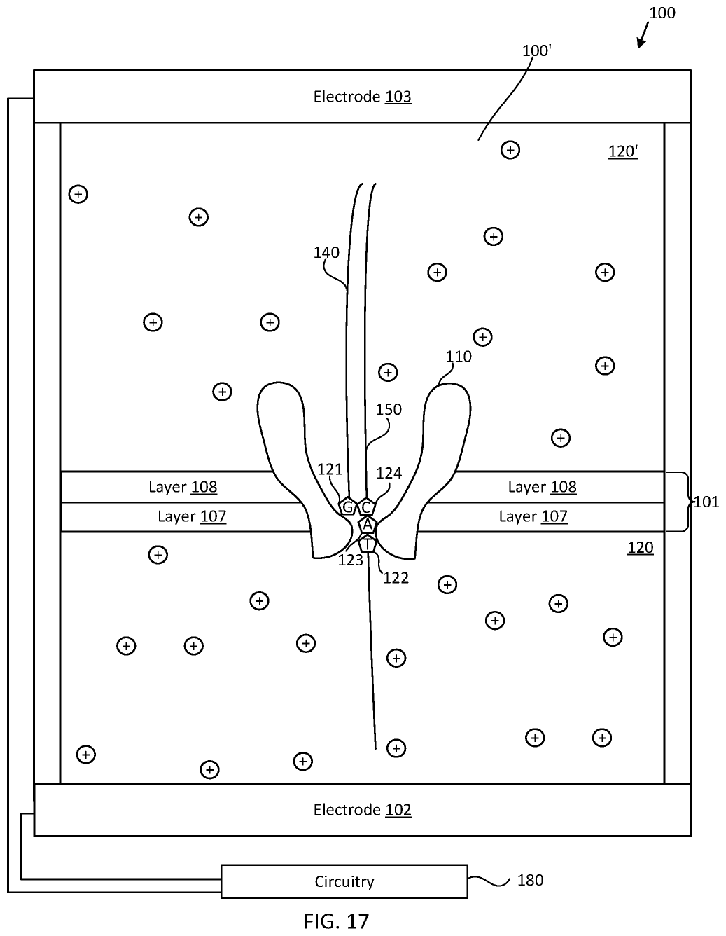

FIG. 17 schematically illustrates a cross-sectional view of another example use of the composition and device of FIG. 1. As illustrated in FIG. 17, device 100 may include fluidic well 100′, barrier 101 which may have a configuration such as described with reference to FIGS. 2A-2C, 14A-14B, 15, and/or 16 (that is, barrier 101 optionally may be suspended using a barrier support, and may include any AB, ABA, or BAB copolymer provided herein), first and second fluids 120, 120′, and nanopore 110 all of which may be configured similarly as described with reference to FIG. 4. In the nonlimiting example illustrated in FIG. 17, a duplex between polynucleotide 140 and polynucleotide 150 is located within nanopore 110 under an applied force, e.g., a bias voltage that circuitry 180 applies between electrode 102 and electrode 103. A combination of bases in the double-stranded portion (here, the base pair GC 121, 124 at the terminal end of the duplex) and bases in the single-stranded portion of polynucleotide 150 (here, bases A and T 123, 122) may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see US Patent Publication No. 2023/0090867 to Mandell et al., the entire contents of which are incorporated by reference herein.

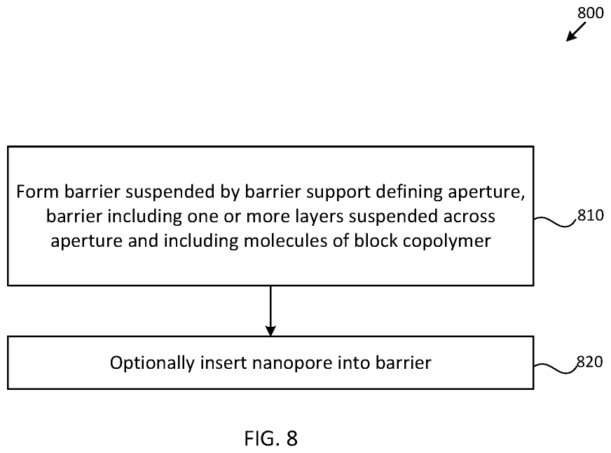

It will be appreciated that compositions and devices such as described with reference to FIGS. 1-7, 14A-14B, 15, 16, and 17 may be prepared in any suitable manner. FIG. 8 illustrates a flow of operations for forming a device such as illustrated in FIG. 1. Method 800 illustrated in FIG. 8 includes forming a barrier between first and second fluids, the barrier being suspended by a barrier support defining an aperture, the barrier including one or more layers suspended across the aperture and comprising molecules of a block copolymer (operation 810). The barrier may be formed using any suitable combination of operations provided herein or otherwise known in the art. For example, forming the barrier may include “painting” as known in the art. Known techniques for painting barriers that are suspended by barrier supports include brush painting (manual), mechanical painting (e.g., using stirring bar), and bubble painting (e.g., using flow through the device).

Each molecule of the block copolymer may include one or more hydrophilic blocks having an approximate length A and one or more hydrophobic blocks having an approximate length B. The one or more hydrophilic blocks may form outer surfaces of the barrier and the hydrophobic blocks being located within the barrier. For example, the barrier may include any AB, ABA, or BAB copolymer provided herein. In some examples, the one or more hydrophobic blocks may include a polymer selected from the group consisting of poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB). For reasons such as explained elsewhere herein, such polymers may be expected to generate suspended membranes with particularly useful qualities for use in nanopore sequencing operations, e.g., such as described with reference to FIGS. 4-7 and 17.

Method 800 optionally also includes inserting a nanopore into the barrier (operation 820). The nanopore may provide contact between the first fluid and the second fluid. The nanopore may be inserted into the barrier using operations such as described elsewhere herein, or otherwise known in the art. Known techniques for inserting a nanopore into a suspended barrier include electroporation, pipette pump cycle, and detergent assisted pore insertion. Tools for forming suspended barriers using synthetic polymers and inserting nanopores in the suspended barriers are commercially available, such as the Orbit 16 TC platform available from Nanion Technologies Inc. (California, USA). It will be appreciated that operation 820 need not necessarily be performed after operation 810, if it is desired to use the barrier without a nanopore.

Illustratively, the block copolymer is an AB diblock copolymer, such as described with reference to FIGS. 2B and 14A-14B. As such, the barrier may have a thickness of approximately 2A+2B. In one nonlimiting example of such a diblock copolymer, the hydrophobic block may be polybutadiene (PBd). Alternatively, the block copolymer may be an ABA triblock copolymer having two hydrophilic blocks and one hydrophobic block. As such, the barrier may have a thickness of approximately 2A+B. In one nonlimiting example of such a triblock copolymer, the hydrophobic block is poly(isobutylene) (PIB) or PDMS. In yet another alternative, the block copolymer is a BAB triblock copolymer having two hydrophobic blocks and one hydrophilic block. As such, the barrier may have a thickness of approximately A+2B.

### WORKING EXAMPLES

The following examples are intended to be purely illustrative, and not limiting of the present invention unless specifically recited in the claims.

### Example 1

PDMS-b-PEO-b-PDMS and PBd-b-PEO performances were assessed in terms of ease of membrane preparation, controlled single vs. multiple nanopore insertion, and stability against osmotic pressure.

All materials were tested on the Orbit-16 instrument from NanION. This tool allows mechanical painting by rotation of a Teflon stirring bar on top of the chip cavities as well as electrical testing of the membrane/pore construct (membrane capacitance measurement, nanopore I/V curve).

### Membrane Painting and Nanopore Insertion

The phospholipid 1,2-diphytanoyl-sn-glycero-3-phosphocholine (DPhPC), which is widely used to form lipid bilayers, PEO₅₀₀-b-PBd₇₀₀ (“P5”, commercially obtained from Polymer Source, Inc. (Quebec, Canada), P41716a-BdEO), and PDMS₅₀₀-b-PEO₄₂₀-b-PDMS₅₀₀ (“P2”, commercially obtained from Polymer Source, Inc, P9091-DMSEODMS) respectively were diluted in octane (5 mg/mL) prior to testing through suspended membrane formation (also called membrane painting) using a support including a circular aperture such as described with reference to FIGS. 14A-14B, 15, and 16. Those 3 materials were selected for comparison because they have similar hydrophilic ratios and relatively low T_{g} and sufficiently low M_{w} of the hydrophobic block to promote pore insertion. More specifically, the T_{g} of PDMS ranges from −130° C. to −120 ° C. The T_{g} of PBd is about −95° C. although it can increase to −12° C. depending on the isomer. Accordingly, in some examples, low T_{g} may be considered as a negative temperature.

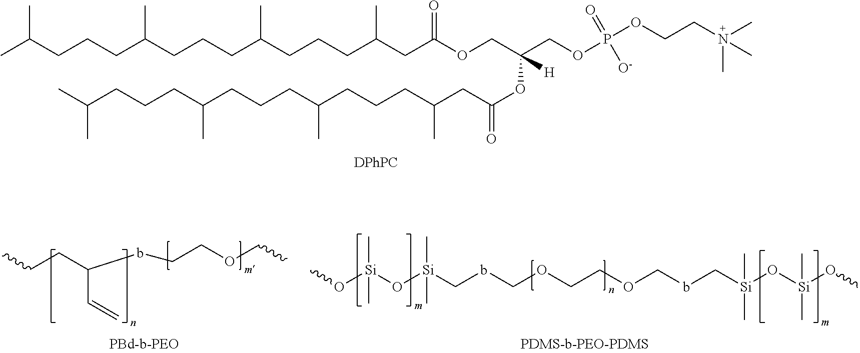

In terms of painting quality, both the AB and BAB polymers required less material and fewer rotations of the stirring bar on top of the cavities to form stable membranes than did DPhPC.

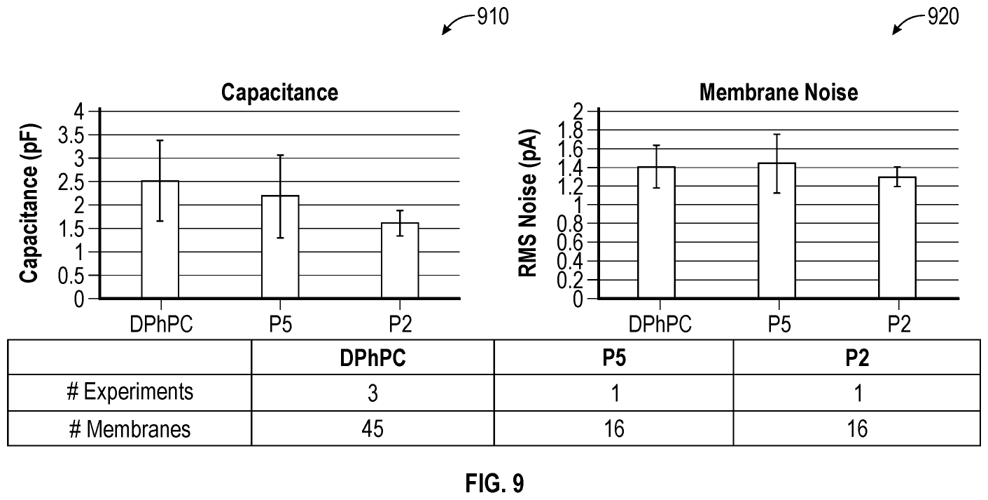

In addition, capacitance values and noise level of the membranes made using the three different compounds were measured. For example, FIG. 9 illustrates plots and a table describing capacitance (plot 910) and noise (plot 920) measured for membranes formed from DPhPC, PEO₅₀₀-b-PBd₇₀₀ (P5) and PDMS₅₀₀-b-PEO₄₂₀-b-PDMS₅₀₀ (P2). As may be understood from plot 910, DPhPC shows the highest capacitance value (around 2.5 pF), where PEO₅₀₀-b-PBd₇₀₀ and PDMS₅₀₀-b-PEO₄₂₀-b-PDMS₅₀₀ show lower capacitance: around 2±0.7 pF and 1.5 respectively. As may be understood from plot 920, membrane noise is similar for the three materials.

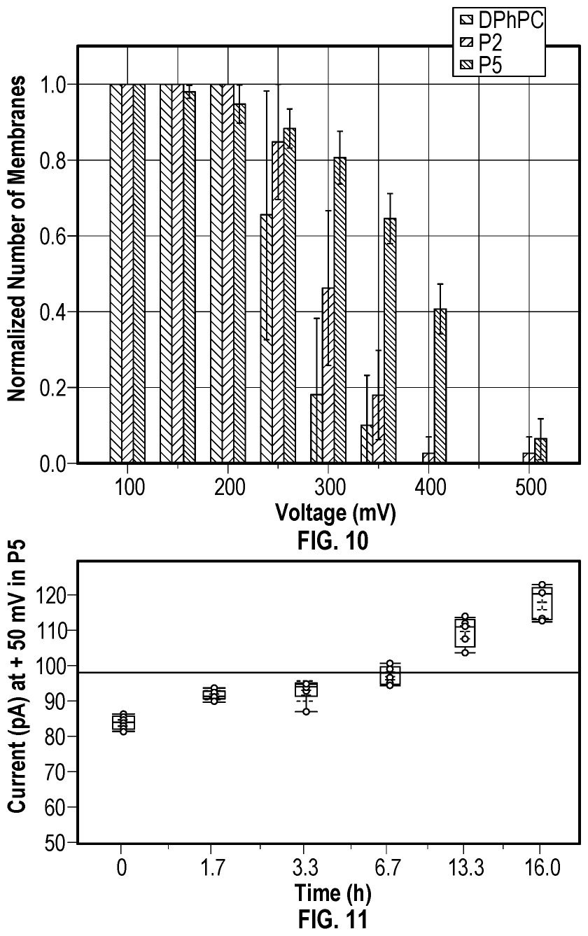

FIG. 10 illustrates a plot of the normalized number of remaining membranes as a function of measured voltage for DPhPC, PEO₅₀₀-b-PBd₇₀₀ (P5), and PDMS₅₀₀-b-PEO₄₂₀-b-PDMS₅₀₀ (P2) membranes. The initial number of membranes (16) were used to normalize the number of membranes remaining at different voltages. Average number of membranes ±STD (n=3). The normalized number of membranes remaining at a given voltage corresponds to the membrane resistance to breakdown voltage. In this test, voltage across the membrane is gradually increased to identify voltage at which membranes start to break. As may be understood from FIG. 10, membranes made from both AB and BAB polymer materials break at much higher voltage compared with DPhPC membrane, highlighting higher their stability.

Another difference between membranes formed using DPhPC and the AB and BAB polymeric materials is how difficult it was to insert a single nanopore into that membrane. For nanopore sequencing such as described with reference to FIGS. 4-7, it is useful to insert a single nanopore in each membrane. However, single nanopore insertion was found to be relatively difficult to achieve with DPhPC, which led to a mixture of membranes having single nanopores and membranes having multiple nanopores. On the other hand, PEO₅₀₀-b-PBd₇₀₀ and PDMS₅₀₀-b-PEO₄₂₀-b-PDMS₅₀₀ were found to provide significantly improved control over single nanopore insertion.

In terms of nanopore properties, MspA nanopore was observed to have a slightly lower current level when captured in the AB or BAB polymer membranes than in the DPhPC membrane. This is believed to be driven by the higher resistance of the polymeric membranes. For example, MspA nanopore in PEO₅₀₀-b-PBd₇₀₀ and PDMS₅₀₀-b-PEO₄₂₀-b-PDMS₅₀₀ membranes were observed to have about 25% higher resistance (lower conductance) than MspA nanopore in DPhPC membrane. MspA nanopore noise level was observed to be comparable between DPhPC and PDMS₅₀₀-b-PEO₄₂₀-b-PDMS₅₀₀ but slightly higher when inserted in PEO₅₀₀-b-PBd₇₀₀ membranes.

To assess stability of an AB polymeric membrane, MspA nanopore/PEO₅₀₀-b-PBd₇₀₀ membrane construct stability in 1M KCl+50 mM HEPES buffer under AC was evaluated. FIG. 11 illustrates a plot of MspA nanopore/PEO₅₀₀-b-PBd₇₀₀ membrane construct stability in 1M KCl+50 mM HEPES buffer. As shown FIG. 11, this nanopore/membrane assembly can remain stable over 16 h. In comparison, MspA inserted into a DPhPC membrane under similar conditions was observed to survive for around 1 hour. The increase of current recorded over time was attributed to buffer evaporation as the system is an open-cell.

### Membrane and Membrane/Pore Construct Stability under Osmotic Pressure

In some configurations, a salt unbalanced set-up may improve performance during nanopore sequencing such as described with reference to FIGS. 4 and 5. For example, lower salt concentration in fluid 120′ (also referred to as the cis side) may help to maintain polymerase activity, and higher salt concentration in fluid 120 (also referred to as the trans side) may help to increase current level. In a nonlimiting example of a balanced salt condition, fluids 120′ and 120 both include 150 mM KCl and 50 mM HEPES. In a nonlimiting example of an unbalanced salt condition, fluid 120′ (cis) includes 50 mM KCl, 50 mM HEPES, and 0.1 mM tris(2-carboxyethyl)phosphine (TCEP); and fluid 120 (trans) includes 300 mM KCl and 50 mM HEPES. In a manner such as described with reference to FIG. 1, such an imbalance in salt concentrations may generate osmotic pressure 191. Both PEO₅₀₀-b-PBd₇₀₀ (P5) and PDMS₅₀₀-b-PEO₄₂₀-b-PDMS₅₀₀ (P2) membranes were observed to survive 500 mM/1000 mM salt conditions. PEO₅₀₀-b-PBd₇₀₀ (P5) was also observed to survive 250 mM/1000 mM unbalanced salt conditions. From this, it may be understood that the present diblock and triblock membranes may be used under an osmotic pressure.

### Example 2

The performance of different copolymers was assessed in terms of ease of membrane preparation, controlled single vs. multiple nanopore insertion, and stability against osmotic pressure.

All materials were tested on the Orbit-16 instrument from NanION. This tool allows mechanical painting by rotation of a Teflon stirring bar on top of the chip cavities as well as electrical testing of the membrane/pore construct (membrane capacitance measurement, nanopore I/V curve).

### Membrane Painting and Nanopore Insertion

Copolymers listed in Table 1 below respectively were dissolved in an octane:butanol (95:5 vol) solvent mixture at a concentration of 5 mg/mL prior to testing through suspended membrane formation (also called membrane painting) using a support including a circular aperture such as described with reference to FIGS. 14A-14B, 15, and 16.

TABLE 1

| Poly- | End | | |
|---|---|---|---|
| mer | group(s) | Label | Structure |
| PEO- b- PIB- b- PEO | —CH₃ | ABA1 | 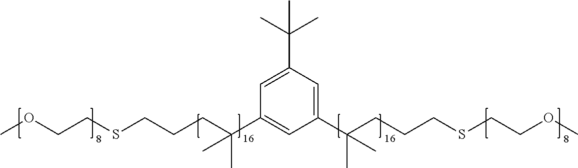 |
| PEO- b- PIB- b- PEO | —COOH | ABA2 | 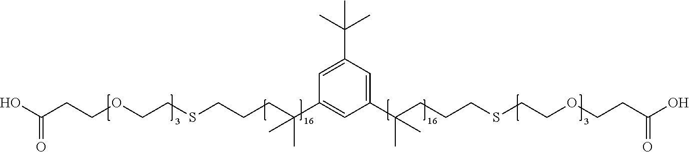 |
| PEO- b- PIB- b- PEO | —COOH | ABA3 | 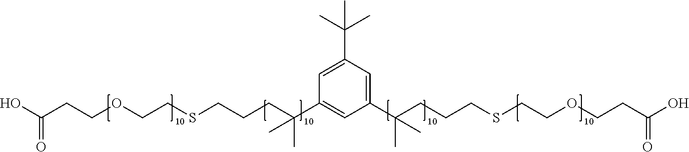 |
| PEO- b- PDMS- b- PEO | —COOH | ABA4 | 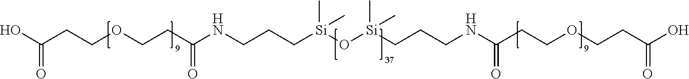 |
| PEO- b- PIB | —CH₃ | AB1 | 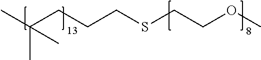 |
| PEO- b- PIB | —CH₃ (on PIB) and —COOH (on PEO) | AB2 | 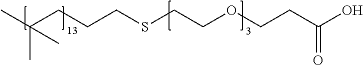 |
| PEO- b- PIB | —CH₃ (on both PIB) and PEO) | AB3 | 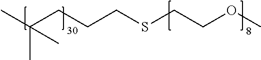 |
| PEO- b- PIB | —CH₃ (on PIB) and —COOH (on PEO) | AB4 | 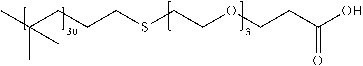 |
| PEO- b- PBD | —CH₃ (on PBD) and —OH (on PEO) | P5 | 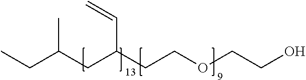 |

Characterization tests were used to extract metrics that are believed to be relevant to the nanopore-sensing application of such membranes. Those metrics fall under categories such as stability (e.g., resilience of membranes/membranes-pore system against stress tests, including accelerated tests, sequencing conditions), throughput (e.g., membrane painting yield, pore insertion and retention yields) and quality (e.g., membrane-pore current and noise level and consistency, SNR, electrical insulation/leakiness of membrane).

The first characterization tests that were carried out focused on success rate in membrane formation (membrane painting yields), membrane resistance to breakdown voltage, biological pore insertion (MspA pores), resulting current and noise of said pore inside of the block copolymer membranes. These helped to assess the performance of the PIB-PEO-based membranes to one another and to membranes formed with other polymers.

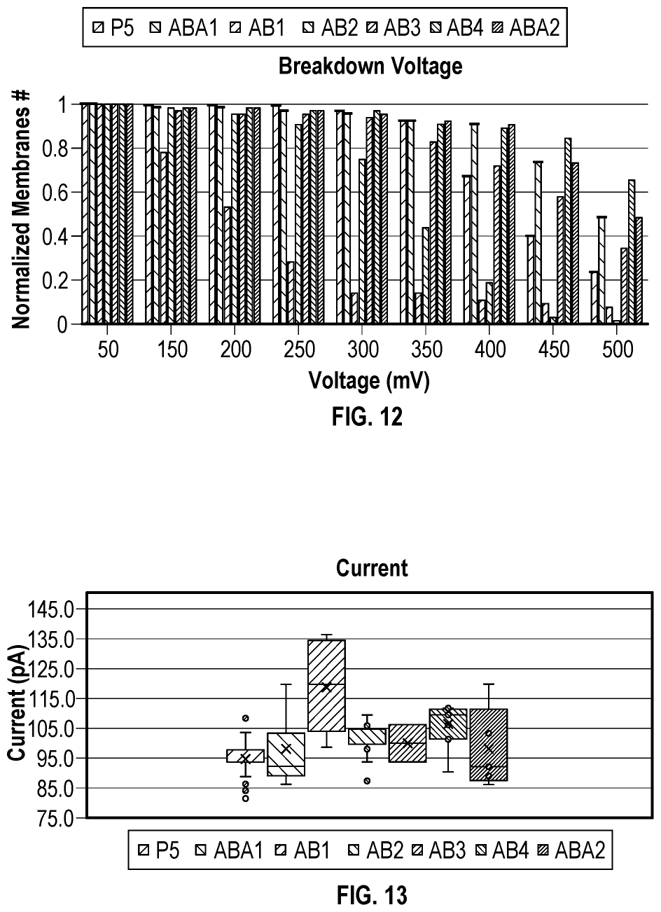

In terms of painting quality, all the PIB-b-PEO block copolymers (AB1, AB2, AB3, AB4) and PEO-b-PIB-b-PEO block copolymers (ABA1, ABA2, ABA3) could be painted to form a suspended membrane. Particularly satisfactory performance was achieved by the PEO-b-PIB-b-PEO block copolymers with an ABA architecture. For example, FIG. 12 illustrates a plot describing the breakdown voltage measured for membranes formed using P5, ABA1, AB1, AB2, AB3, AB4, and ABA2. For the membrane formed using P5, it may be seen in FIG. 12 that at voltages of about 300 mV and below, the normalized number of membranes which remained substantially intact ranged from about 1.0 at 0 V to about 0.95 at 300 mV; and that at voltages of about 350 mV and above, the normalized number of membranes decreased from about 0.9 at 350 mV to about 0.22 at 500 mV. For the membrane formed using ABA1, it may be seen in FIG. 12 that at voltages of about 300 mV and below, the normalized number of membranes which remained substantially intact ranged from about 1.0 at 0 V to about 0.95 at 300 mV; and that at voltages of about 350 mV and above, the normalized number of membranes decreased from about 0.9 at 350 mV to about 0.5 at 500 mV. For the membrane formed using ABA2, it may be seen in FIG. 12 that at voltages of about 300 mV and below, the normalized number of membranes which remained substantially intact ranged from about 1.0 at 0 V to about 0.95 at 300 mV; and that at voltages of about 350 mV and above, the normalized number of membranes decreased from about 0.9 at 350 mV to about 0.5 at 500 mV.

For the membrane formed using AB1, it may be seen in FIG. 12 that at voltages of about 300 mV and below, the normalized number of membranes which remained substantially intact ranged from about 1.0 at 0 V to about 0.16 at 300 mV; and that at voltages of about 350 mV and above, the normalized number of membranes decreased from about 0.16 at 350 mV to about 0.04 at 500 mV. For the membrane formed using AB2, it may be seen in FIG. 12 that at voltages of about 300 mV and below, the normalized number of membranes which remained substantially intact ranged from about 1.0 at 0 V to about 0.75 at 300 mV; and that at voltages of about 350 mV and above, the normalized number of membranes decreased from about 0.45 at 350 mV to about 0.01 at 500 mV. For the membrane formed using AB3, it may be seen in FIG. 12 that at voltages of about 300 mV and below, the normalized number of membranes which remained substantially intact ranged from about 1.0 at 0 V to about 0.9 at 300 mV; and that at voltages of about 350 mV and above, the normalized number of membranes decreased from about 0.85 at 350 mV to about 0.035 at 500 mV. For the membrane formed using AB4, it may be seen in FIG. 12 that at voltages of about 300 mV and below, the normalized number of membranes which remained substantially intact ranged from about 1.0 at 0 V to about 0.97 at 300 mV; and that at voltages of about 350 mV and above, the normalized number of membranes decreased from about 0.9 at 350 mV to about 0.65 at 500 mV.

As may be understood from the plot shown in FIG. 12, the ABA block copolymers ABA1 and ABA2, and the AB4 block copolymer, showed particularly high resilience to the breakdown voltage stress test.

Another difference between membranes is how difficult it was to insert a single nanopore into that membrane. For nanopore sequencing such as described with reference to FIGS. 4-7 and 17, it is useful to insert a single nanopore in each membrane.

FIG. 13 illustrates a plot of MspA nanopore/membrane construct stability in 1M KCl+50 mM HEPES buffer. For the membrane formed using P5 with an MspA nanopore inserted therein, it may be seen in FIG. 13 that at a voltage of 100 mV, the membrane-pore construct had a current which ranged between about 92 pA and about 97 pA. For the membrane formed using ABA1 with an MspA nanopore inserted therein, it may be seen in FIG. 13 that at a voltage of 100 mV, the membrane-pore construct had a current which ranged between about 89 pA and about 104 pA. For the membrane formed using ABA2 with an MspA nanopore inserted therein, it may be seen in FIG. 13 that at a voltage of 100 mV, the membrane-pore construct had a current which ranged between about 87 pA and about 110 pA. For the membrane formed using AB1 with an MspA nanopore inserted therein, it may be seen in FIG. 13 that at a voltage of 100 mV, the membrane-pore construct had a current which ranged between about 104 pA and about 135 pA. For the membrane formed using AB2 with an MspA nanopore inserted therein, it may be seen in FIG. 13 that at a voltage of 100 mV, the membrane-pore construct had a current which ranged between about 100 pA and about 105 pA. For the membrane formed using AB3 with an MspA nanopore inserted therein, it may be seen in FIG. 13 that at a voltage of 100 mV, the membrane-pore construct had a current which ranged between about 94 pA and about 107 pA. For the membrane formed using AB4 with an MspA nanopore inserted therein, it may be seen in FIG. 13 that at a voltage of 100 mV, the membrane-pore construct had a current which ranged between about 96 pA and about 110 pA.

The ABA2 and ABA3 membranes were identified as having particularly good performance. A notable improvement shown in ABA2 is the enhancement of membrane resilience. A notable improvement shown in ABA3 is the enhancement of the insertion and retention of single MspA nanopores into the membrane, with a lower variability. Various properties of the membranes are shown in Table 2 below.

TABLE 2

| Category | Property | ABA4 | | ABA2 | | ABA3 | |
|---|---|---|---|---|---|---|---|
| Membrane | Membrane survival rate at | 95% | | 100% | | 100% | |
| quality | 450 mV (%) | | | | | | |
| MspA | Open Pore Current (50 um | 104 | pA | 103 | pA | 104 | pA |
| nanopore | wells, 100 mV) (pA) | | | | | | |
| current | Membrane-Pore (MP) | 2.13 | pA | 1.46 | pA | 1.62 | pA |
| | RMS noise (pA) | | | | | | |
| | Pore + membrane SNR | 49 | | 71 | | 64 | |
| Yields | Membrane painting yield | >95% | | >95% | | >95% | |
| | (number of membranes | | | | | | |
| | formed, divided by number | | | | | | |
| | of attempts to form | | | | | | |
| | membranes) (%) | | | | | | |
| | Pore insertion voltage | 500-850 | mV | 800-1000 | mV | 350-450 | mV |
| | (mV) | | | | | | |
| | Single Pore (SP) | >95% | | ~90% | | >95% | |
| | percentage (after insertion | | | | | | |
| | step) (%) | | | | | | |
| | SP Survival Rate under | >95% | | >95% | | >95% | |
| | flush (3 × 250 uL) (%) | | | | | | |
| MspA | Open Pore current | 36.18 | pA | 35.02 | pA | 36.04 | pA |
| nanopore | (400 mM KCl, 50 mV, | | | | | | |
| current + | 50 um wells) (pA) | | | | | | |
| consistency | SP current std dev (within | 1.93 | pA | 0.8 | pA | 0.4 | pA |
| | 1 exp) (±pA) | | | | | | |
| Resilience | 700 to 1200 mV/10 us- | ~65% | | ~95% | | ~95% | |
| against | membrane survival at 1200 | | | | | | |
| biochemistry | mV (%) | | | | | | |
| | 700 to 1200 mV/10 us- | >1200 | mV | >1200 | mV | >1200 | mV |
| | voltage at 50% membrane | | | | | | |
| | survival (mV) | | | | | | |
| | 700 to 1200 mV/10 us- | 1000 | mV | 1000 | mV | 900 | mV |
| | voltage at 50% membrane | | | | | | |
| | and single pore survival | | | | | | |
| | (mV) | | | | | | |

Membrane quality was measured by membrane survival rate at 450 mV of current across the barrier. Both ABA2 and ABA3 had a 100% survival rate, and ABA4 had an approximately 95% survival rate.

The current through the membrane when an MspA nanopore was inserted was also measured. The open pore current was measured at a voltage across the barrier of 100 mV. ABA2 and ABA3 had similar currents of 103 pA and 104 pA, respectively, and ABA4 had a current of 104 pA. The root mean square (RMS) average of the current noise through the barrier was also measured in a similar fashion, after inserting the MspA nanopore into the membrane. ABA2 and ABA3 had similar RMS current noise averages of 1.46 pA and 1.62 pA, respectively, and ABA4 had a RNS current noise of 2.13 pA. The signal-to-noise ratio (SNR) of current through the barrier was also measured in a similar fashion. ABA2 and ABA3 had SNRs of 71 and 64, respectively, and ABA4 had a SNR of 49.

Various yield percentages were also measured. ABA2, ABA3, and ABA4 all had a membrane painting yield of greater than 95%. The voltage required to insert an MspA nanopore into the membrane was also measured in a similar fashion. ABA2 required a voltage between about 800 mV and about 1000 mV, while ABA3 required a voltage between about 350 mV and 450 mV, and ABA4 required a voltage between about 500 mV and about 850 mV. After a nanopore insertion step was carried out, the percentage of membranes containing an MspA nanopore was about 90% for ABA2, greater than 95% for ABA3, and greater than 95% of ABA4. The percentage of single-pore membranes surviving a flush was also measured in a similar fashion. Specifically, the membranes were flushed with 250 μL of fluid three times before the survival rate was determined. Both ABA2 and ABA3 had survival rates of greater than 95%, as did ABA4.

The tightness of the open pore current spread of MspA nanopores within the membrane were also measured. Specifically, the current was measured 400 mM KCl and applying a current of 50 mV across the membrane. ABA2 and ABA3 had open pore currents of 35.02 pA and 36.04 pA, respectively, and ABA4 had an open pore current of 36.18 pA. The standard deviation of the current was measured in a similar fashion. ABA2 and ABA3 had standard deviations of 0.8 pA and 0.4 pA, respectively, and ABA4 had a standard deviation of 1.93 pA. The resilience of the barriers was also measured. The membrane survival rate was measured after the barrier was subjected to a waveform which was made of a train of positive voltage micro pulses, spaced by negative voltage periods at −100 mV for 100 ms. The train of positive voltage pulses had a total of 20 pulses, with duration of 10 μs. The spacings between them had a set duration value of 30 ms and a voltage held at +50 mV. During a first cycle, the waveform was applied continuously for a period of 5 minutes and the magnitude of the pulses kept at +700 mV. In further applied cycles (applied again for 5 mins each), the pulsing intensity was increased from +700 mV to +1200 mV in 100 mV steps, for a total of six cycles. Both ABA2 and ABA3 had a survival rate, after application of the described waveform, of about 95%, while ABA4 had a survival rate of about 65%. In the same experiment/test, the voltage at which 50% of the membranes survived after being subjected to said waveforms was determined. For ABA2, ABA3, and AB4, the voltage was determined to be greater than 1200 mV. In another test/experiment, the same waveform cycle/test was repeated, but using membranes after having a single MspA pore inserted. For ABA2 and ABA4, the voltage was determined to be about 1000 mV, while for ABA3, the voltage was determined to be about 900 mV.

Based on the different metrics selected to assess fitness for use in polymeric membranes for nanopore sequencing applications, these results demonstrate that the performance of membranes including PIB as the hydrophobic B block are particularly suitable for use in such applications. For example, the different metrics indicate that ABA and AB copolymers using PIB as the hydrophobic B block, particularly those including —COOH as the end group, can form membranes with useful properties for nanopore sequencing applications, including relatively high membrane formation yield, relatively high pore insertion and retention yield, relatively high stability under sequencing conditions, and relatively good electrical properties for high read quality. The inventors believe that such properties are likely to be correlated with ease of flow-cell fabrication, instrument throughout, instrument/chip reliability, and high read accuracy, which may be important to commercial manufacture and use of nanopore sequencing devices, e.g., such as described with reference to FIGS. 4-7 and 17.

Certain aspects of the present subject matter are expressed in the following clauses. The present invention is not intended to be limited to such clauses, unless expressly recited in the claims.
Clause 1. A barrier between first and second fluids, the barrier comprising:
a first layer comprising a first plurality of molecules of a diblock copolymer,
   each molecule of the diblock copolymer comprising a hydrophobic block coupled to a hydrophilic block; and
a second layer comprising a second plurality of molecules of the diblock copolymer,
   the hydrophilic blocks of the first plurality of molecules forming a first outer surface of the barrier,
   the hydrophilic blocks of the second plurality of molecules forming a second outer surface of the barrier, and
   the hydrophobic blocks of the first and second pluralities of molecules contacting one another within the barrier.
      Clause 2. The barrier of clause 1, wherein the hydrophilic block is approximately of length A, the hydrophobic block is approximately of length B, the first and second layers each have a thickness of approximately A+B, and the barrier has a thickness of approximately 2A+2B.
      Clause 3. The barrier of clause 2, wherein the length A is about 2 repeating units to about 100 repeating units.
      Clause 4. The barrier of clause 2 or clause 3, wherein the length A is about 5 repeating units to about 15 repeating units.
      Clause 5. The barrier of any one of clauses 2 to 4, wherein the length B is about 2 repeating units to about 100 repeating units.
      Clause 6. The barrier of any one of clauses 2 to 5, wherein the length B is about 2 repeating units to about 50 repeating units.
      Clause 7. The barrier of any one of clauses 2 to 6, wherein the length B is about 10 repeating units to about 15 repeating units.
      Clause 8. The barrier of any one of clauses 1 to 7, wherein the hydrophilic block includes a polymer selected from the group consisting of: N-vinyl pyrrolidone, polyacrylamide, zwitterionic polymer, polypeptide, and poly(ethylene oxide) (PEO).
      Clause 9. The barrier of clause 8, wherein the polyacrylamide is selected from the group consisting of: poly(N-isopropyl acrylamide) (PNIPAM), charged polyacrylamide, and phosphoric acid functionalized polyacrylamide.
      Clause 10. The barrier of any one of clauses 1 to 9, wherein the hydrophobic block includes a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).
      Clause 11. The barrier of any one of clauses 1 to 10, each of the molecules further comprising a linker coupling the hydrophobic block to the hydrophilic block.
      Clause 12. The barrier of clause 11, wherein the linker includes at least one moiety selected from the group consisting of: an amide, a thioether (sulfide), a succinic group, a maleic group, a methylene, an ether, and a product of a click reaction.
      Clause 13. A device comprising:
a fluidic well holding first and second fluids;
the barrier of any one of clauses 1 to 12 disposed between the first and second fluids in the fluidic well; and
a nanopore disposed within the barrier and providing an aperture fluidically coupling the first fluid to the second fluid.
   Clause 14. The device of clause 13, wherein the first fluid has a first composition and the second fluid has a second composition that is different from the first composition.
   Clause 15. The device of clause 14, wherein the first fluid has a first concentration of a salt and the second fluid has a second concentration of the salt that is different from the first concentration.
   Clause 16. A method of making the device of any one of clauses 13 to 15, the method comprising:
forming the barrier in the fluidic well; and
inserting the nanopore within the barrier.
   Clause 17. The method of clause 16, wherein forming the barrier comprises painting.
   Clause 18. The method of clause 17, wherein the painting comprises a technique selected from the group consisting of brush painting, mechanical painting, and bubble painting.
   Clause 19. The method of any one of clauses 16 to 18, wherein inserting the nanopore comprises a technique selected from the group consisting of electroporation, pipette pump cycle, and detergent assisted pore insertion.
   Clause 20. A method of making a diblock copolymer, the method comprising:
polymerizing a plurality of hydrophilic monomers to form a hydrophilic polymer;
forming an initiator at a terminal end of the hydrophilic polymer; and
using the initiator to polymerize a plurality of hydrophobic monomers to form a hydrophobic polymer coupled to the hydrophilic polymer.
   Clause 21. A method of making a diblock copolymer, the method comprising:
polymerizing a plurality of hydrophobic monomers to form a hydrophobic polymer;
forming an initiator at a terminal end of the hydrophobic polymer; and
using the initiator to polymerize a plurality of hydrophilic monomers to form a hydrophilic polymer coupled to the hydrophobic polymer.
   Clause 22. The method of clause 20 or clause 21, wherein the initiator comprises bromine, iodine, or chlorine.
   Clause 23. The method of clause 20 or clause 21, wherein the initiator comprises a chain transfer agent.
   Clause 24. The method of clause 23, wherein the chain transfer agent comprises S-1-dodecyl-S′-(α,α′-dimethyl-α″-acetic acid) trithiocarbonate (DDMAT).
   Clause 25. The method of any one of clauses 20 to 24, wherein the hydrophilic polymer comprises a polymer selected from the group consisting of: N-vinyl pyrrolidone, polyacrylamide, zwitterionic polymer, polypeptide, and poly(ethylene oxide) (PEO).
   Clause 26. The method of clause 25, wherein the polyacrylamide is selected from the group consisting of: poly(N-isopropyl acrylamide) (PNIPAM), charged polyacrylamide, and phosphoric acid functionalized polyacrylamide.
   Clause 27. The method of any one of clauses 20 to 26, wherein the hydrophobic polymer comprises a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).
   Clause 28. The method of any one of clauses 20 to 27, wherein the hydrophobic polymer is coupled to the hydrophilic polymer via a linker.
   Clause 29. The method of clause 28, wherein the linker is selected from the group consisting of an amide, a thioether (sulfide), a succinic group, a maleic group, a methylene, an ether, and a product of a click reaction.
   Clause 30. A method of making a diblock copolymer, the method comprising:
polymerizing a plurality of hydrophilic monomers to form a hydrophilic polymer;
polymerizing a plurality of hydrophobic monomers to form a hydrophobic polymer; and
coupling the hydrophilic polymer to the hydrophobic polymer.
   Clause 31. The method of clause 30, wherein a terminal end of the hydrophilic polymer comprises a first reactive moiety, and a terminal end of the hydrophobic polymer comprises a second reactive moiety that reacts with the first reactive moiety to couple the hydrophilic polymer to the hydrophobic polymer.
   Clause 32. The method of clause 31, wherein one of the first and second reactive moieties comprises an azide and the other of the first and second reactive moieties comprises an alkyne.
   Clause 33. The method of clause 31, wherein one of the first and second reactive moieties comprises a thiol and the other of the first and second reactive moieties comprises an alkene.
   Clause 34. The method of clause 31, wherein one of the first and second reactive moieties comprises a thiol and the other of the first and second reactive moieties comprises an alkyne.
   Clause 35. The method of clause 31, wherein one of the first and second reactive moieties comprises an amine and the other of the first and second reactive moieties comprises N-hydroxysuccinimide.
   Clause 36. The method of any one of clauses 30 to 35, wherein the hydrophilic polymer comprises a polymer selected from the group consisting of: N-vinyl pyrrolidone, polyacrylamide, zwitterionic polymer, polypeptide, and poly(ethylene oxide) (PEO).
   Clause 37. The method of clause 36, wherein the polyacrylamide is selected from the group consisting of: poly(N-isopropyl acrylamide) (PNIPAM), charged polyacrylamide, and phosphoric acid functionalized polyacrylamide.
   Clause 38. The method of any one of clauses 30 to 37, wherein the hydrophobic polymer comprises a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).
   Clause 39. The method of any one of clauses 30 to 38, wherein the hydrophobic polymer is coupled to the hydrophilic polymer via a linker.
   Clause 40. The method of clause 39, wherein the linker is selected from the group consisting of an amide, a thioether (sulfide), a succinic group, a maleic group, a methylene, an ether, and a product of a click reaction.
   Clause 41. A barrier between first and second fluids, the barrier comprising:
a first layer comprising a first plurality of molecules of a triblock copolymer,
each molecule of the triblock copolymer comprising first and second hydrophobic blocks and a hydrophilic block disposed between the first and second hydrophobic blocks; and
a second layer comprising a second plurality of molecules of the triblock copolymer,
the hydrophilic blocks of the first plurality of molecules forming a first outer surface of the barrier,
the hydrophilic blocks of the second plurality of molecules forming a second outer surface of the barrier, and
the hydrophobic blocks of the first and second pluralities of molecules contacting one another within the barrier.
   Clause 42. The barrier of clause 41, wherein the hydrophilic block is approximately of length A, each hydrophobic block is approximately of length B, the first and second layers each have a thickness of approximately A/2+B, and the barrier has a thickness of approximately A+2B.
   Clause 43. The barrier of clause 42, wherein the length A is about 2 repeating units to about 100 repeating units.
   Clause 44. The barrier of clause 42 or clause 43, wherein the length A is about 2 repeating units to about 50 repeating units.
   Clause 45. The barrier of any one of clauses 42 to 44, wherein the length A is about 5 repeating units to about 20 repeating units.
   Clause 46. The barrier of any one of clauses 42 to 45, wherein the length A is about 13 repeating units to about 19 repeating units.
   Clause 47. The barrier of any one of clauses 42 to 45, wherein the length A is about 7 repeating units to about 13 repeating units.
   Clause 48. The barrier of clause 42 or clause 43, wherein the length A is about 2 repeating units to about 30 repeating units.
   Clause 49. The barrier of any one of clauses 42 to 43 or clause 48, wherein the length A is about 2 repeating units to about 15 repeating units.
   Clause 50. The barrier of any one of clauses 42 to 43 or clauses 48 to 49, wherein the length A is about 7 repeating units to about 11 repeating units.
   Clause 51. The barrier of any one of clauses 42 to 43 or clauses 48 to 49, wherein the length A is about 2 repeating units to about 5 repeating units.
   Clause 52. The barrier of any one of clauses 42 to 51, wherein the length B is about 2 repeating units to about 100 repeating units.
   Clause 53. The barrier of any one of clauses 42 to 52, wherein the length B is about 2 repeating units to about 50 repeating units.
   Clause 54. The barrier of any one of clauses 42 to 53, wherein the length B is about 2 repeating units to about 15 repeating units.
   Clause 55. The barrier of any one of clauses 42 to 54, wherein the length B is about 2 repeating units to about 5 repeating units.
   Clause 56. The barrier of any one of clauses 42 to 54, wherein the length B is about 7 repeating units to about 13 repeating units.
   Clause 57. The barrier of any one of clauses 42 to 52, wherein the length B is about 25 repeating units to about 45 repeating units.
   Clause 58. The barrier of any one of clauses 42 to 52 or clause 57, wherein the length B is about 30 repeating units to about 40 repeating units.
   Clause 59. The barrier of any one of clauses 42 to 52 or clauses 57 to 58, wherein the length B is about 35 repeating units to about 40 repeating units.
   Clause 60. The barrier of any one of clauses 42 to 52 or clauses 57 to 58, wherein the length B is about 30 repeating units to about 37 repeating units.
   Clause 61. The barrier of any one of clauses 41 to 60, wherein the hydrophilic block comprises a polymer selected from the group consisting of: N-vinyl pyrrolidone, polyacrylamide, zwitterionic polymer, polypeptide, and poly(ethylene oxide) (PEO).
   Clause 62. The barrier of clause 61, wherein the polyacrylamide is selected from the group consisting of: poly(N-isopropyl acrylamide) (PNIPAM), charged polyacrylamide, and phosphoric acid functionalized polyacrylamide.
   Clause 63. The barrier of any one of clauses 41 to 62, wherein the hydrophobic blocks each comprise a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).
   Clause 64. The barrier of any one of clauses 41 to 63, each of the molecules of the first and second plurality of molecules further comprising a linker coupling each hydrophobic block to the hydrophilic block.
   Clause 65. The barrier of clause 64, wherein the linker is selected from the group consisting of an amide, a thioether (sulfide), a succinic group, a maleic group, a methylene, an ether, and a product of a click reaction.
   Clause 66. A device comprising:
a fluidic well holding first and second fluids;
the barrier of any one of clauses 41 to 65 disposed between the first and second fluids in the fluidic well; and
a nanopore disposed within the barrier and providing an aperture fluidically coupling the first fluid to the second fluid.
   Clause 67. The device of clause 66, wherein the first fluid has a first composition and the second fluid has a second composition that is different from the first composition.
   Clause 68. The device of clause 67, wherein the first fluid has a first concentration of a salt and the second fluid has a second concentration of the salt that is different from the first concentration.
   Clause 69. A method of making the device of any one of clauses 66 to 68, the method comprising:
forming the barrier in the fluidic well; and
inserting the nanopore within the barrier.
   Clause 70. The method of clause 69, wherein forming the barrier comprises painting.
   Clause 71. The method of clause 70, wherein the painting comprises a technique selected from the group consisting of brush painting, mechanical painting, and bubble painting.
   Clause 72. The method of any one of clauses 69 to 71, wherein inserting the nanopore comprises a technique selected from the group consisting of electroporation, pipette pump cycle, and detergent assisted pore selection.
   Clause 73. A method of making a triblock copolymer, the method comprising:
polymerizing a plurality of hydrophilic monomers to form a hydrophilic polymer;
forming initiators at respective terminal ends of the hydrophilic polymer; and
using the initiators to polymerize a plurality of hydrophobic monomers to form a hydrophobic polymer coupled to each terminal end of the hydrophilic polymer.
   Clause 74. The method of clause 73, wherein the initiators each comprise bromine, iodine, or chlorine.
   Clause 75. The method of clause 73 or clause 74, wherein the initiators each comprise a chain transfer agent.
   Clause 76. The method of clause 75, wherein the chain transfer agent comprises S-1-dodecyl-S′-(α,α′-dimethyl-α″-acetic acid) trithiocarbonate (DDMAT).
   Clause 77. The method of any one of clauses 73 to 76, wherein the hydrophilic polymer comprises a polymer selected from the group consisting of: N-vinyl pyrrolidone, polyacrylamide, zwitterionic polymer, polypeptide, and poly(ethylene oxide) (PEO).
   Clause 78. The method of clause 77, wherein the polyacrylamide is selected from the group consisting of: poly(N-isopropyl acrylamide) (PNIPAM), charged polyacrylamide, and phosphoric acid functionalized polyacrylamide.
   Clause 79. The method of any one of clauses 73 to 78, wherein the hydrophobic polymers each comprise a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).
   Clause 80. The method of any one of clauses 73 to 79, wherein the hydrophobic polymers are each coupled to the hydrophilic polymer via a linker.
   Clause 81. The method of clause 80, wherein the linker is selected from the group consisting of an amide, a thioether (sulfide), a succinic group, a maleic group, a methylene, an ether, and a product of a click reaction.
   Clause 82. A method of making a triblock copolymer, the method comprising:
polymerizing a plurality of hydrophilic monomers to form a hydrophilic polymer having terminal ends;
polymerizing a plurality of hydrophobic monomers to form first and second hydrophobic polymers; and
coupling the first and second hydrophobic polymers to respective terminal ends of the hydrophilic polymer.
   Clause 83. The method of clause 82, wherein a terminal end of each of the first and second hydrophobic polymers comprises a first reactive moiety, and each terminal end of the hydrophilic polymer comprises a second reactive moiety that reacts with the first reactive moiety to couple the first and second hydrophobic polymers to the hydrophilic polymer.
   Clause 84. The method of clause 83, wherein one of the first and second reactive moieties comprises an azide and the other of the first and second reactive moieties comprises an alkyne.
   Clause 85. The method of clause 83, wherein one of the first and second reactive moieties comprises a thiol and the other of the first and second reactive moieties comprises an alkene.
   Clause 86. The method of clause 83, wherein one of the first and second reactive moieties comprises a thiol and the other of the first and second reactive moieties comprises an alkyne.
   Clause 87. The method of clause 83, wherein one of the first and second reactive moieties comprises an amine and the other of the first and second reactive moieties comprises N-hydroxysuccinimide.
   Clause 88. The method of any one of clauses 82 to 87, wherein the hydrophilic polymer comprises a polymer selected from the group consisting of: N-vinyl pyrrolidone, polyacrylamide, zwitterionic polymer, polypeptide, and poly(ethylene oxide) (PEO).
   Clause 89. The method of clause 88, wherein the polyacrylamide is selected from the group consisting of: poly(N-isopropyl acrylamide) (PNIPAM), charged polyacrylamide, and phosphoric acid functionalized polyacrylamide.
   Clause 90. The method of any one of clauses 82 to 89, wherein the first and second hydrophobic polymers each comprise a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene).
   Clause 91. The method of any one of clauses 82 to 90, wherein the first and second hydrophobic polymers are each coupled to the hydrophilic polymer via a linker.
   Clause 92. The method of clause 91, wherein the linker is selected from the group consisting of an amide, a thioether (sulfide), a succinic group, a maleic group, a methylene, an ether, and a product of a click reaction.
   Clause 93. A barrier between first and second fluids, the barrier comprising:
a layer comprising a plurality of molecules of a triblock copolymer,
each molecule of the triblock copolymer comprising first and second hydrophilic blocks and a hydrophobic block disposed between the first and second hydrophilic blocks,
the first hydrophilic blocks of the first plurality of molecules forming a first outer surface of the barrier,
the second hydrophilic blocks of the plurality of molecules forming a second outer surface of the barrier, and
the hydrophobic blocks of the plurality of molecules contacting one another within the barrier.
   Clause 94. The barrier of clause 93, wherein each hydrophilic block is approximately of length A, the hydrophobic block is approximately of length B, the layer has a thickness of approximately 2A+B, and the barrier has a thickness of approximately 2A+B.
   Clause 95. The barrier of clause 94, wherein the length A is about 2 repeating units to about 100 repeating units.
   Clause 96. The barrier of clause 94 or clause 95, wherein the length A is about 2 repeating units to about 50 repeating units.
   Clause 97. The barrier of any one of clauses 94 to 96, wherein the length A is about 5 repeating units to about 20 repeating units.
   Clause 98. The barrier of any one of clauses 94 to 97, wherein the length A is about 13 repeating units to about 19 repeating units.
   Clause 99. The barrier of any one of clauses 94 to 97, wherein the length A is about 7 repeating units to about 13 repeating units.
   Clause 100. The barrier of clause 94 or clause 95, wherein the length A is about 2 repeating units to about 30 repeating units.
   Clause 101. The barrier of any one of clauses 94 to 95 or clause 100, wherein the length A is about 2 repeating units to about 15 repeating units.
   Clause 102. The barrier of any one of clauses 94 to 95 or clauses 100 to 101, wherein the length A is about 7 repeating units to about 11 repeating units.
   Clause 103. The barrier of any one of clauses 94 to 95 or clauses 100 to 101, wherein the length A is about 2 repeating units to about 5 repeating units.
   Clause 104. The barrier of any one of clauses 94 to 103, wherein the length B is about 2 repeating units to about 100 repeating units.
   Clause 105. The barrier of any one of clauses 94 to 104, wherein the length B is about 2 repeating units to about 50 repeating units.
   Clause 106. The barrier of any one of clauses 94 to 105, wherein the length B is about 2 repeating units to about 15 repeating units.
   Clause 107. The barrier of any one of clauses 94 to 106, wherein the length B is about 2 repeating units to about 5 repeating units.
   Clause 108. The barrier of any one of clauses 94 to 106, wherein the length B is about 7 repeating units to about 13 repeating units.
   Clause 109. The barrier of any one of clauses 94 to 104, wherein the length B is about 25 repeating units to about 45 repeating units.
   Clause 110. The barrier of any one of clauses 94 to 104 or clause 109, wherein the length B is about 30 repeating units to about 40 repeating units.
   Clause 111. The barrier of any one of clauses 94 to 104 or clauses 109 to 110, wherein the length B is about 35 repeating units to about 40 repeating units.
   Clause 112. The barrier of any one of clauses 94 to 104 or clauses 109 to 110, wherein the length B is about 30 repeating units to about 37 repeating units.
   Clause 113. The barrier of any one of clauses 93 to 112, wherein each hydrophilic block comprises a polymer selected from the group consisting of: N-vinyl pyrrolidone, polyacrylamide, zwitterionic polymer, polypeptide, and poly(ethylene oxide) (PEO).
   Clause 114. The barrier of clause 113, wherein the polyacrylamide is selected from the group consisting of: poly(N-isopropyl acrylamide) (PNIPAM), charged polyacrylamide, and phosphoric acid functionalized polyacrylamide.
   Clause 115. The barrier of any one of clauses 93 to 114, wherein the hydrophobic block comprises a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).
   Clause 116. The barrier of any one of clauses 93 to 115, each of the molecules of the plurality of molecules further comprising a linker coupling the hydrophobic block to each of the hydrophilic blocks.
   Clause 117. The barrier of clause 116, wherein the linker is selected from the group consisting of an amide, a thioether (sulfide), a succinic group, a maleic group, a methylene, an ether, and a product of a click reaction.
   Clause 118. A device comprising:
a fluidic well holding first and second fluids;
the barrier of any one of clauses 93 to 117 disposed between the first and second fluids in the fluidic well; and
a nanopore disposed within the barrier and providing an aperture fluidically coupling the first fluid to the second fluid.
   Clause 119. The device of clause 118, wherein the first fluid has a first composition and the second fluid has a second composition that is different from the first composition.
   Clause 120. The device of clause 119, wherein the first fluid has a first concentration of a salt and the second fluid has a second concentration of the salt that is different from the first concentration.
   Clause 121. A method of making the device of clause 118, the method comprising:
forming the barrier in the fluidic well; and
inserting the nanopore within the barrier.
   Clause 122. The method of clause 121, wherein forming the barrier comprises painting.
   Clause 123. The method of clause 122, wherein the painting comprises a technique selected from the group consisting of brush painting, mechanical painting, and bubble painting.
   Clause 124. The method of any one of clauses 121 to 123, wherein inserting the nanopore comprises a technique selected from the group consisting of electroporation, pipette pump cycle, and detergent assisted pore selection.
   Clause 125. A method of making a triblock copolymer, the method comprising:
polymerizing a plurality of hydrophobic monomers to form a hydrophobic polymer;
forming initiators at respective terminal ends of the hydrophobic polymer; and
using the initiators to polymerize a plurality of hydrophilic monomers to form a hydrophilic polymer coupled to each terminal end of the hydrophobic polymer.
   Clause 126. The method of clause 125, wherein the initiators each comprise bromine, iodine, or chlorine.
   Clause 127. The method of clause 125 or clause 126, wherein the initiators each comprise a chain transfer agent.
   Clause 128. The method of clause 127, wherein the chain transfer agent comprises S-1-dodecyl-S′-(α,α′-dimethyl-α″-acetic acid) trithiocarbonate (DDMAT).
   Clause 129. The method of any one of clauses 125 to 128, wherein the hydrophilic polymers each comprise a polymer selected from the group consisting of: N-vinyl pyrrolidone, polyacrylamide, zwitterionic polymer, polypeptide, and poly(ethylene oxide) (PEO).
   Clause 130. The method of clause 129, wherein the polyacrylamide is selected from the group consisting of: poly(N-isopropyl acrylamide) (PNIPAM), charged polyacrylamide, and phosphoric acid functionalized polyacrylamide.
   Clause 131. The method of any one of clauses 125 to 130, wherein the hydrophobic polymer comprises a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).
   Clause 132. The method of any one of clauses 125 to 131, wherein the hydrophobic polymer is coupled to the hydrophilic polymers via a linker.
   Clause 133. The method of clause 132, wherein the linker is selected from the group consisting of an amide, a thioether (sulfide), a succinic group, a maleic group, a methylene, an ether, and a product of a click reaction.
   Clause 134. A method of making a triblock copolymer, the method comprising:
polymerizing a plurality of hydrophobic monomers to form a hydrophobic polymer having terminal ends;
polymerizing a plurality of hydrophilic monomers to form first and second hydrophilic polymers; and
coupling the first and second hydrophilic polymers to respective terminal ends of the hydrophobic polymer.
   Clause 135. The method of clause 134, wherein a terminal end of each of the first and second hydrophilic polymers comprises a first reactive moiety, and each terminal end of the hydrophobic polymer comprises a second reactive moiety that reacts with the first reactive moiety to couple the first and second hydrophilic polymers to the hydrophobic polymer.
   Clause 136. The method of clause 135, wherein one of the first and second reactive moieties comprises an azide and the other of the first and second reactive moieties comprises an alkyne.
   Clause 137. The method of clause 135, wherein one of the first and second reactive moieties comprises a thiol and the other of the first and second reactive moieties comprises an alkene.
   Clause 138. The method of clause 135, wherein one of the first and second reactive moieties comprises a thiol and the other of the first and second reactive moieties comprises an alkyne.
   Clause 139. The method of clause 135, wherein one of the first and second reactive moieties comprises an amine and the other of the first and second reactive moieties comprises N-hydroxysuccinimide.
   Clause 140. The method of any one of clauses 134 to 139, wherein the first and second hydrophilic polymers each comprise a polymer selected from the group consisting of: N-vinyl pyrrolidone, polyacrylamide, zwitterionic polymer, polypeptide, and poly(ethylene oxide) (PEO).
   Clause 141. The method of clause 140, wherein the polyacrylamide is selected from the group consisting of: poly(N-isopropyl acrylamide) (PNIPAM), charged polyacrylamide, and phosphoric acid functionalized polyacrylamide.
   Clause 142. The method of any one of clauses 134 to 141, wherein the hydrophobic polymer comprises a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene).
   Clause 143. The method of any one of clauses 134 to 142, wherein the first and second hydrophilic polymers are coupled to the hydrophobic polymer via a linker.
   Clause 144. The method of clause 143, wherein the linker is selected from the group consisting of an amide, a thioether (sulfide), a succinic group, a maleic group, a methylene, an ether, and a product of a click reaction.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A barrier between first and second fluids, the barrier being suspended by a barrier support defining an aperture, the barrier comprising:
one or more layers suspended across the aperture and comprising molecules of a block copolymer,
each molecule of the block copolymer comprising one or more hydrophilic blocks having an approximate length A and one or more hydrophobic blocks having an approximate length B,
the hydrophilic blocks forming outer surfaces of the barrier and the hydrophobic blocks being located within the barrier,
the hydrophobic blocks comprising a polymer selected from the group consisting of poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

2. The barrier of claim 1, wherein the block copolymer is a diblock copolymer.

3. The barrier of claim 2, wherein the hydrophobic block is polybutadiene (PBd).

4. The barrier of claim 2, wherein the barrier has a thickness of approximately 2A+2B.

5. The barrier of claim 1, wherein the block copolymer is a triblock copolymer having two hydrophilic blocks and one hydrophobic block.

6. The barrier of claim 5, wherein the hydrophobic block is poly(isobutylene) (PIB).

7. The barrier of claim 5, wherein the barrier has a thickness of approximately 2A+B.

8. The barrier of claim 1, wherein the block copolymer is a triblock copolymer having two hydrophobic blocks and one hydrophilic block.

9. The barrier of claim 8, wherein the barrier has a thickness of approximately A+2B.

10. The barrier of claim 1, further comprising a nanopore disposed therein and providing contact between the first fluid and the second fluid.

11. A barrier between first and second fluids, the barrier being suspended by a barrier support defining an aperture, the barrier comprising:
one or more layers suspended across the aperture and comprising molecules of a block copolymer,
each molecule of the block copolymer comprising first and second hydrophilic blocks and a hydrophobic block disposed between the first and second hydrophilic blocks,
the first and second hydrophilic blocks forming outer surfaces of the barrier and the hydrophobic blocks being located within the barrier; and
a nanopore disposed within the barrier and providing contact between the first fluid and the second fluid.

12. The barrier of claim 11, wherein the first and second hydrophilic blocks are approximately of length A, the hydrophobic block is approximately of length B, at least a portion of the one or more layers has a thickness of approximately 2A+B, and the barrier has a thickness of approximately 2A+B.

13. The barrier of claim 12, wherein the length A is about 2 repeating units to about 100 repeating units.

14. The barrier of claim 12, wherein the length B is about 2 repeating units to about 100 repeating units.

15. The barrier of claim 11, wherein the hydrophobic block comprises a polymer selected from the group consisting of poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

16. The barrier of claim 11, wherein the first and second hydrophilic blocks comprise a polymer selected from the group consisting of: N-vinyl pyrrolidone, polyacrylamide, zwitterionic polymer, polypeptide, and poly(ethylene oxide) (PEO).

17. The barrier of claim 11, wherein the hydrophobic block comprises a polymer having a glass transition temperature (T_{g}) of less than about 0° C.

18. The barrier of claim 11, wherein at least one of the first and second hydrophilic blocks comprises a moiety selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen.

19. The barrier of claim 18, wherein the moiety of the first hydrophilic block comprises a methyl group or a carboxyl group, and wherein the moiety of the second hydrophilic block comprises a methyl group or a carboxyl group.

20. The barrier of claim 11, wherein the molecules further comprise a first linker coupling the first hydrophilic block to a first end of the hydrophobic block, and a second linker coupling the second hydrophilic block to a second end of the hydrophobic block.

21. A barrier between first and second fluids, the barrier comprising:
a first layer comprising a first plurality of molecules of a diblock copolymer,
each molecule of the diblock copolymer comprising a hydrophobic block coupled to a hydrophilic block; and
a second layer comprising a second plurality of molecules of the diblock copolymer,
the hydrophilic blocks of the first plurality of molecules forming a first outer surface of the barrier,
the hydrophilic blocks of the second plurality of molecules forming a second outer surface of the barrier, and
the hydrophobic blocks of the first and second pluralities of molecules contacting one another within the barrier.

22. The barrier of claim 21, wherein the hydrophilic block is approximately of length A, the hydrophobic block is approximately of length B, the first and second layers each have a thickness of approximately A+B, and the barrier has a thickness of approximately 2A+2B.

23. The barrier of claim 22, wherein the length A is about 2 repeating units to about 100 repeating units.

24. The barrier of claim 22, wherein the length B is about 2 repeating units to about 100 repeating units.

25. The barrier of claim 21, wherein the hydrophilic block includes a polymer selected from the group consisting of: N-vinyl pyrrolidone, polyacrylamide, zwitterionic polymer, polypeptide, and poly(ethylene oxide) (PEO).

26. The barrier of claim 21, wherein the hydrophobic block includes a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

27. The barrier of claim 21, each of the molecules further comprising a linker coupling the hydrophobic block to the hydrophilic block.

28. The barrier of claim 27, wherein the linker includes at least one moiety selected from the group consisting of: an amide, a thioether (sulfide), a succinic group, a maleic group, a methylene, an ether, and a product of a click reaction.

29. The barrier of claim 21, wherein the first and second hydrophilic blocks each include a moiety independently selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen.

30. The barrier of claim 21, further comprising a nanopore disposed within the barrier and providing an aperture fluidically coupling the first fluid to the second fluid.

31. A barrier between first and second fluids, the barrier comprising:
a first layer comprising a first plurality of molecules of a triblock copolymer,
each molecule of the triblock copolymer comprising first and second hydrophobic blocks and a hydrophilic block disposed between the first and second hydrophobic blocks; and
a second layer comprising a second plurality of molecules of the triblock copolymer,
the hydrophilic blocks of the first plurality of molecules forming a first outer surface of the barrier,
the hydrophilic blocks of the second plurality of molecules forming a second outer surface of the barrier, and
the hydrophobic blocks of the first and second pluralities of molecules contacting one another within the barrier.

32. The barrier of claim 31, wherein the hydrophilic block is approximately of length A, each hydrophobic block is approximately of length B, the first and second layers each have a thickness of approximately A/2+B, and the barrier has a thickness of approximately A+2B.

33. The barrier of claim 32, wherein the length A is about 2 repeating units to about 100 repeating units.

34. The barrier of claim 32, wherein the length B is about 2 repeating units to about 100 repeating units.

35. The barrier of claim 31, wherein the hydrophilic block comprises a polymer selected from the group consisting of: N-vinyl pyrrolidone, polyacrylamide, zwitterionic polymer, polypeptide, and poly(ethylene oxide) (PEO).

36. The barrier of claim 31, wherein the hydrophobic blocks each comprise a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB).

37. The barrier of claim 31, each of the molecules of the first and second plurality of molecules further comprising a linker coupling each hydrophobic block to the hydrophilic block.

38. The barrier of claim 37, wherein the linker is selected from the group consisting of an amide, a thioether (sulfide), a succinic group, a maleic group, a methylene, an ether, and a product of a click reaction.

39. The barrier of claim 31, wherein the hydrophilic block includes a moiety selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen.

40. The barrier of claim 31, further comprising a nanopore disposed within the barrier and providing an aperture fluidically coupling the first fluid to the second fluid.

41-50. (canceled)
